(19) United States
(12) Patent Application Publication
(10) Pub. No.: US 2013/0164289 A1
(43) Pub. Date: Jun. 27, 2013

(54) HUMAN CYTOMEGALOVIRUS VACCINE

(71) Applicants: Michael McVoy, Richmond, VA (US); Stuart Adler, Richmond, VA (US); Xiaohong Cui, Quinton, VA (US)

(72) Inventors: Michael McVoy, Richmond, VA (US); Stuart Adler, Richmond, VA (US); Xiaohong Cui, Quinton, VA (US)

(73) Assignee: Virginia Commonwealth University, Richmond, VA (US)

(21) Appl. No.: 13/820,700

(22) PCT Filed: Sep. 9, 2011
(86) PCT No.: PCT/US2011/051008

§ 371 Date: Mar. 4, 2013

Related U.S. Application Data
(60) Provisional application No. 61/381,280, filed on Sep. 9, 2010.

(51) Int. Cl. C07K 14/045; A61K 39/245; C07K 16/18; C07K 16/08; A61K 39/395; C07K 14/02; A61K 39/385

(52) U.S. Cl. CPC C07K 14/045; C07K 14/02; A61K 39/245; A61K 39/385; C07K 16/088; A61K 39/39533; C07K 16/18
USPC 424/134.1; 530/300; 530/324; 530/350; 424/230.1; 424/186.1; 424/192.1; 424/199.1; 424/200.1; 424/159.1; 530/387.3; 435/235.1

(57) ABSTRACT

Combination peptides, polypeptides and proteins that elicit high titer neutralizing antibodies against cytomegalovirus (CMV) are provided. The combination peptides, polypeptides and proteins encompass epitopes located within the UL130 and UL131 components of the gH/gL/UL128-131 protein complex, in particular, epitopes located within amino acid residues 27-46 of UL130 and amino acid residues 90-106 of UL131. The combination peptides, polypeptides and proteins, and the nucleic acids encoding them, may be used in vaccines, and as diagnostic and research tools.

### BACKGROUND OF THE INVENTION

1. Field of the Invention

The invention generally relates to peptides, polypeptides and proteins which, when administered to a human, elicit the production of neutralizing antibodies against cytomegalovirus (CMV). In particular, the invention provides combination peptides, polypeptides and proteins (e.g. chimeric and fusion constructs, carrier complexes, etc.) which include 1) multiple copies of peptides corresponding to one or both of amino acid residues 27-46 of CMV protein UL130 and/or amino acid residues 90-106 of CMV protein UL131; or 2) at least one peptide corresponding to amino acid residues 27-46 of CMV protein UL130 or amino acid residues 90-106 of CMV protein UL131, plus at least one other proteinaceous entity.

2. Background of the Invention

Congenital cytomegalovirus (CMV) infections are a frequent cause of birth defects and illness in transplant patients and immunocompromised individuals, e.g. those suffering from AIDS. Studies evaluating active or passive immunization to prevent or treat these infections have shown that CMV hyperimmune globulin, which contains CMV-reactive antibodies induced by natural infection, appears effective for treating and preventing both congenital and transplant-associated infections [1, 2]. Active immunization with either a live attenuated virus or a glycoprotein B subunit vaccine prevents CMV disease associated with renal transplantation [3] and reduces the risk of primary maternal CMV infection [4]. For both active and passive immunization, neutralizing activity is probably essential.

Only two candidate vaccines, Towne and gB/MF59, have completed phase II efficacy trials. In an experimental challenge study, immunization with Towne was protective against both infection and disease caused by challenge with pathogenic Toledo strain and several studies found that vaccination with Towne prior to renal transplantation is effective in preventing severe post-transplantation CMV disease. However, in a small phase II clinical trial, a low dose of Towne vaccine failed to show protection against infection of seronegative mothers who had children actively shedding CMV.

The gB/MF59 vaccine is a protein subunit vaccine comprised of a transmembrane-deleted version of CMV gB protein administered with the proprietary oil and water adjuvant MF59. The gB/MF59 vaccine induces high levels of fibroblast entry neutralizing antibodies in humans and has been shown to be safe and well tolerated in both adults and toddlers. A recent phase II double-blind placebo-controlled trial of the gB/MF59 vaccine revealed a 50% efficacy in inducing sterilizing immunity. As this vaccine induces potent antibody responses but very weak T-cell responses, the partial efficacy provided by the gB/MF59 vaccine is thought to be primarily antibody-mediated. This is the first CMV vaccine to show any protective efficacy, and while 50% protection is a significant achievement, it falls short of the 80-90% desired for most vaccines.

In the past CMV neutralizing activity was measured using fibroblasts as target cells. However, recent experiments demonstrate that antibodies to epitopes within a pentameric complex of gH, gL, UL128, UL130, and UL131 (gH/gL/UL128-131) neutralize entry into endothelial, epithelial, and other cell types but have no effect on fibroblast entry [5-8]. This is because the gH/gL/UL128-131 complex is essential for entry into endothelial and epithelial cells but is fully dispensable for fibroblast entry [5, 9, 10]. Indeed, mutations causing loss of UL128, UL130, or UL131 expression are sufficient to eliminate endothelial tropism [9] and occur within relatively few passages in fibroblasts [11]. Natural infection elicits very high titer neutralizing antibodies specific for epithelial cell entry and it has been proposed that antibodies against gH/gL/UL128-131 epitopes may comprise a significant component of this activity [7, 8, 12]. In contrast, epithelial entry neutralizing titers induced by the Towne live attenuated vaccine or the gB subunit vaccine were 28- and 15-fold lower, respectively, than those induced by natural infection [12]. These results suggest that vaccine efficacy may be improved using antigens that elicit high titer epithelial entry neutralizing antibodies.

United States patent application 20090081230 to Lanzavecchia et al. (the complete contents of which is hereby incorporated by reference) describes neutralizing antibodies and antibody fragments having high potency in neutralizing hCMV. The antibodies and antibody fragments are specific for a combination of hCMV proteins UL130 and UL131A, or for a combination of hCMV proteins UL128, UL130 and UL131A. However, the antibodies were raised against entire UL128, UL130 and UL131A proteins and particular epitopes were not identified.

U.S. Pat. No. 7,704,510 to Shenk et al. (the complete contents of which is hereby incorporated by reference) describes immunogenic compositions and prophylactic or therapeutic vaccines for use in protecting and treating human cytomegalovirus (CMV). Subunit vaccines comprising at least one cytomegalovirus (CMV) protein or fragment thereof, selected from pUL128, pUL130, or a complex that includes pUL128 or pUL130, are described, as are therapeutic antibodies reactive against a CMV protein complex comprising pUL128 or pUL130, as well as diagnostic and screening methods using the subunits.

Ryckman et al., (Journal of Virology, 82; 60-70) generated antibodies against peptides corresponding to positions 27-46 of UL130 and 90-106 of UL131 in order to characterize interactions between the proteins of the CMV gH/gL/UL128-131 complex. The antibodies were used to detect UL130 and UL131 by immunoblot and to precipitate UL130 or UL131 to identify their interactions with other proteins in the complex. Antibodies raised against UL130 or UL131 peptides were confirmed to interact specifically with their intended target proteins (UL130 and UL131) but were not evaluated for immunologically relevant functions such as the capacity to neutralize CMV entry.

There is an ongoing need to identify potent vaccinogens that elicit protective, neutralizing immune responses to CMV, and to develop CMV vaccines that achieve protection levels of at least 80-90%.

### SUMMARY OF THE INVENTION

The invention provides peptides, polypeptides and proteins that elicit high titer neutralizing antibodies which prevent the entry of CMV into epithelial cells. The peptides, polypeptides and proteins are “combinations” in that they include either: 1) multiple copies of one or the other or both of the peptide sequence located at residues 27-46 of UL130 and the peptide sequence located at residues 90-106 of UL131; or 2) at least one peptide sequence located at residues 27-46 of UL130 or the peptide sequence located at residues 90-106 of UL131, plus one other proteinaceous entity. The other entity may be, for example, a carrier protein, a targeting sequence, an immunogeninc sequence that is not from CMV, or another immunogenic sequence that is from CMV (e.g., gB protein and/or a genetically engineered form thereof), etc. The invention provides vaccines and/or immunogenic compositions which comprise the combination peptides, polypeptides and proteins; methods of using the compositions, antibodies to the combination peptides, polypeptides and proteins; and nucleic acids encoding the combination peptides, polypeptides and proteins. In one embodiment, a vaccine composition also includes at least one other CMV immunogen, e.g. the gB protein and/or a genetically engineered form thereof. The peptides described herein may be fused, conjugated, or otherwise attached to said “other CMV immunogen”.

The invention provides combination peptides, polypeptides and proteins comprising, in one embodiment: I. a plurality of copies of one or both of a) amino acid residues 27-46 of a UL130 cytomegalovirus (CMV) protein; and b) amino acid residues 90-106 of a UL131 cytomegalovirus (CMV) protein; or, in another embodiment, II. i) one or more copies of one or both of a. amino acid residues 27-46 of a UL130 cytomegalovirus (CMV) protein; and b. amino acid residues 90-106 of a UL131 cytomegalovirus (CMV) protein; and ii) an additional proteinaceous entity. The combination peptide, polypeptide or protein is not full-length UL130 CMV or full-length UL131 protein. In one embodiment, the combination peptide, polypeptide or protein comprises a sequence: X₁WX₂TLTANX₃NPSPPWSKLTY (SEQ ID NO: 7) wherein X₁═S or P; X₂═S or F; and X₃=Q or K. In another embodiment, the combination peptide, polypeptide or protein of claim 2, wherein said combination peptide, polypeptide or protein comprises an amino acid sequence selected from the group consisting of and as set forth in or represented by: SWSTLTANQNPSPPWSKLTY (SEQ ID NO: 1); PWSTLTANQNPSPPWSKLTY (SEQ ID NO: 2); PWFTLTANQNPSPPWSKLTY (SEQ ID NO:3); PWSTLTANKNPSPPWSKLTY (SEQ ID NO:4); and PWSTLTANQNPSPLWSKLTY (SEQ ID NO: 5). In yet another embodiment, the combination peptide, polypeptide or protein comprises an amino acid sequence SDFRRQNRRGGTNKRTT (SEQ ID NO: 6). In some embodiments, the additional proteinaceous entity is selected from the group consisting of: a carrier protein suitable for administration to humans, a recombinant hepatitis B core protein, and a red blood cell targeting protein. In other embodiments, the combination peptide, polypeptide or protein further comprises linker or spacer sequence located between the copies of the one or both of amino acid residues 27-46 of a UL130 cytomegalovirus (CMV) protein and amino acid residues 90-106 of a UL131 cytomegalovirus (CMV) protein.

The invention also provides a composition for eliciting a neutralizing immune response against cytomegalovirus (CMV) in a human subject in need thereof, the composition comprising, in one embodiment, combination peptides, polypeptides or proteins comprising: I. a plurality of copies of one or both of a) amino acid residues 27-46 of a UL130 cytomegalovirus (CMV) protein; and b) amino acid residues 90-106 of a UL131 cytomegalovirus (CMV) protein; or, in another embodiment, H. i) one or more copies of one or both of a. amino acid residues 27-46 of a UL130 cytomegalovirus (CMV) protein; and b. amino acid residues 90-106 of a UL131 cytomegalovirus (CMV) protein; and ii) an additional proteinaceous entity. The combination peptide, polypeptide or protein is not full-length UL130 CMV or full-length UL131 protein. In one embodiment, the combination peptide, polypeptide or protein comprises a sequence: X₁WX₂TLTANX₃NPSPPWSKLTY (SEQ ID NO: 7) wherein X₁═S or P; X₂═S or F; and X₃=Q or K. In another embodiment, the combination peptide, polypeptide or protein of claim 2, wherein said combination peptide, polypeptide or protein comprises an amino acid sequence selected from the group consisting of and as set forth in or represented by: SWSTLTANQNPSPPWSKLTY (SEQ ID NO: 1); PWSTLTANQNPSPPWSKLTY (SEQ ID NO: 2); PWFTLTANQNPSPPWSKLTY (SEQ ID NO:3); PWSTLTANKNPSPPWSKLTY (SEQ ID NO:4); and PWSTLTANQNPSPLWSKLTY (SEQ ID NO: 5). In yet another embodiment, the combination peptide, polypeptide or protein comprises an amino acid sequence SDFRRQNRRGGTNKRTT (SEQ ID NO: 6). In some embodiments, the additional proteinaceous entity is selected from the group consisting of: a carrier protein suitable for administration to humans, a recombinant hepatitis B core protein, and a red blood cell targeting protein. In other embodiments, the combination peptide, polypeptide or protein further comprises linker or spacer sequence located between the copies of the one or both of amino acid residues 27-46 of a UL130 cytomegalovirus (CMV) protein and amino acid residues 90-106 of a UL131 cytomegalovirus (CMV) protein. In one embodiment, the composition further comprises CMV glycoprotein B or a genetically engineered version thereof. In another embodiment, the composition also comprises an adjuvant.

The invention also provides a method of eliciting a neutralizing immune response against cytomegalovirus (CMV) in a human subject in need thereof. The method comprises the step of administering to the human subject a composition comprising one or more polypeptides which comprise: I. a plurality of copies of one or both of a) amino acid residues 27-46 of a UL130 cytomegalovirus (CMV) protein; and b) amino acid residues 90-106 of a UL131 cytomegalovirus (CMV) protein; or, in another embodiment, II. i) one or more copies of one or both of a. amino acid residues 27-46 of a UL130 cytomegalovirus (CMV) protein; and b. amino acid residues 90-106 of a UL131 cytomegalovirus (CMV) protein; and ii) an additional proteinaceous entity. The combination peptide, polypeptide or protein is not full-length UL130 CMV or full-length UL131 protein. In one embodiment, the combination peptide, polypeptide or protein comprises a sequence: X₁WX₂TLTANX₃NPSPPWSKLTY (SEQ ID NO: 7) wherein X₁═S or P; X₂═S or F; and X₃=Q or K. In another embodiment, the combination peptide, polypeptide or protein of claim 2, wherein said combination peptide, polypeptide or protein comprises an amino acid sequence selected from the group consisting of and as set forth in or represented by: SWSTLTANQNPSPPWSKLTY (SEQ ID NO: 1); PWSTLTANQNPSPPWSKLTY (SEQ ID NO: 2); PWFTLTANQNPSPPWSKLTY (SEQ ID NO:3); PWSTLTANKNPSPPWSKLTY (SEQ ID NO:4); and PWSTLTANQNPSPLWSKLTY (SEQ ID NO: 5). In yet another embodiment, the combination peptide, polypeptide or protein comprises an amino acid sequence SDFRRQNRRGGTNKRTT (SEQ ID NO: 6). In some embodiments, the additional proteinaceous entity is selected from the group consisting of: a carrier protein suitable for administration to humans, a recombinant hepatitis B core protein, and a red blood cell targeting protein. In other embodiments, the combination peptide, polypeptide or protein further comprises linker or spacer sequence located between the copies of the one or both of amino acid residues 27-46 of a UL130 cytomegalovirus (CMV) protein and amino acid residues 90-106 of a UL131 cytomegalovirus (CMV) protein. In one embodiment, the composition further comprises CMV glycoprotein B or a genetically engineered version thereof. In another embodiment, the composition also comprises an adjuvant. In one embodiment, the immune response that is elicited is production of neutralizing antibodies against CMV. In this embodiment, the neutralizing antibodies prevent entry of CMV into epithelial cells, e.g. oral or genital mucosal epithelial cells.

The invention also provides a method of preventing cytomegalovirus (CMV) entry into cells. The method comprises the step of exposing the CMV to neutralizing antibodies which bind specifically to one or both of i) one or more epitopes within amino acid residues 27-46 of a UL130 cytomegalovirus (CMV) protein; and ii) one or more epitopes within amino acid residues 90-106 of a UL131 CMV protein.

The invention further provides a nucleic acid vaccine composition for vaccinating a subject against cytomegalovirus (CMV). The nucleic acid vaccine composition comprises i) a nucleic acid expression system comprising a nucleic acid that encodes at least one copy of one or more of: a peptide, polypeptide or protein comprising amino acid residues 27-46 of a UL130 CMV protein, or a functional variant thereof; and a peptide, polypeptide or protein comprising amino acid residues 90-106 of a UL131 CMV protein, or a functional variant thereof, the nucleic acid being operably linked to a promoter; and ii) a physiologically acceptable carrier. The nucleic acid is selected from DNA and RNA. In one embodiment, the nucleic acid expression system is a recombinant plasmid vector. In another embodiments, the nucleic acid expression system is a recombinant viral or bacterial expression vector.

The invention further provides a method of generating neutralizing antibodies against cytomegalovirus (CMV). The method comprises the step of administering to an antibody producing mammal, a composition comprising at least one polypeptide which comprises: I. a plurality of copies of one or both of a) amino acid residues 27-46 of a UL130 cytomegalovirus (CMV) protein; and b) amino acid residues 90-106 of a UL131 cytomegalovirus (CMV) protein; or, in another embodiment, II. i) one or more copies of one or both of a. amino acid residues 27-46 of a UL130 cytomegalovirus (CMV) protein; and b. amino acid residues 90-106 of a UL131 cytomegalovirus (CMV) protein; and ii) an additional proteinaceous entity; and a physiologically compatible carrier.

The invention further provides cytomegalovirus (CMV) neutralizing antibodies generated by administering, to an antibody producing mammal, a composition comprising one or more polypeptides which comprise I. a plurality of copies of one or both of a) amino acid residues 27-46 of a UL130 cytomegalovirus (CMV) protein; and b) amino acid residues 90-106 of a UL131 cytomegalovirus (CMV) protein; or, in another embodiment, II. i) one or more copies of one or both of a. amino acid residues 27-46 of a UL130 cytomegalovirus (CMV) protein; and b. amino acid residues 90-106 of a UL131 cytomegalovirus (CMV) protein; and ii) an additional proteinaceous entity; and a physiologically compatible carrier.

### BRIEF DESCRIPTION OF THE DRAWINGS

FIG. 1. Matching inocula of CMV variants HB15-t178b and BADrUL131-Y4 were 10-fold serial diluted and added to wells of 24-well plates containing confluent cultures of the indicated cells. Cultures were monitored by fluorescence microscopy and photographed on the days indicated after infection. Numbers on the left indicate infectious viral dose (pfu/well).

FIG. 2. The indicated dilutions of sera from six CMV seropositive and two CMV seronegative human subjects, postimmune rabbit anti-peptide sera, and corresponding preimmune rabbit sera were incubated with 500 pfu of virus BADrUL131-Y4 for one hour, then used to infect ARPE-19 epithelial cells. In the bottom row (top panel) equal amounts of rabbit anti-UL130 and anti-UL131 were mixed before being assayed as for the other sera. No serum was added to the wells in the right-most column (top panel). Representative micrographs were taken with a fixed exposure four days post infection.

FIG. 3. IC₅₀ values for the same six seropositive human sera shown in FIG. 2, the postimmune rabbit anti-UL130 and -UL131 sera, and the mixture of anti-UL130/UL131 sera were calculated using GFP fluorescence values measured from triplicate assays seven days post infection. Error bars indicate standard errors of the means.

FIG. 4. Replicate amounts of BADrUL131-Y4 were mixed with no serum (ø) or 1:20 dilutions of the indicated rabbit anti-peptide antisera. After one hour incubation the mixtures were added to confluent cultures containing the indicated cells and the cultures were monitored daily by fluorescence microscopy. Photographs shown are from day seven post infection.

FIG. 5A-C. A, VLP assembled in vitro from HBcAg. B, Structural model of HBcAg monomer showing the loop (aa 77-82) that forms the spikes of the VLP. C, SDS-PAGE analysis of purified BEE6 protein under reducing and non-reducing conditions. Marker molecular weights are indicated on the left; arrow indicates the monomeric BEE6 protein.

FIG. 6: Amino acid sequence of chimeric protein BEE6 (SEQ ID NO: 8) with corresponding nucleic acid sequence (SEQ ID NO: 9). The UL130 peptide sequence is boxed.

### DETAILED DESCRIPTION

Peptide sequences have been identified within the UL130 and UL131 protein components of the CMV gH/gL/UL128-131 complex which are sufficient to elicit production of neutralizing antibodies against CMV. The sequences are located at positions 27-46 of UL130 and 90-106 of UL131. When the antigenic peptides are administered to a mammal, the mammal produces antibodies which prevent CMV infection of cells, especially oral and genital mucosal epithelial cells. Without being bound by theory, it is believed that antibodies to the peptides prevent the entry of CMV into potential host cells, thus blocking this route of infection. This is particularly advantageous because the most common route of entry of CMV into the body is through oral and genital mucosal epithelial cells. The ability to block this route of transmission prevents or slows the development of CMV infection in individuals to whom the peptides, or antibodies to the peptides, have been administered, and thus also slows or prevents the transmission of CMV between individuals. Accordingly, combination peptides, polypeptides and proteins which include these antigenic sequences are described herein, as are methods of using the same.

The following definitions are used throughout:

By “cytomegalovirus” herein we mean the viral genus of the viral group known as Herpesviridae or herpesviruses that infects humans. This virus is also known as human CMV or human herpesvirus-5 (HHV-5), and in the literature, is usually abbreviated as CMV, hCMV or HCMV. These three abbreviations may be used interchangeably herein. A large number of strains of HCMV are known, including but not limited to TR, Towne, AD169, Toledo, Merlin, TB40, Davis, etc. The amino acid and nucleic acid sequences disclosed herein should be considered only as examples of those which are present in HCMV strains, and homologs or variants of these sequences in other strains may also be used in the practice of the invention.

By “UL130 peptide” and the “UL131 peptide” we mean peptides with amino acid sequences that are the same as residues 27-46 of CMV UL130 protein and residues 90-106 of CMV UL131 protein, respectively. In the art, CMV proteins may also be designated using the letter “p” in front of the protein name, as in, “pUL130” and “pUL131”. Both conventions (with and without the “p”) may be used herein. In addition, UL131 may also be designated UL131A.

By “antigen” we mean a substance that stimulates production of antibodies. The substance is usually a protein, polypeptide or peptide, and in nature, antigens are frequently surface exposed, i.e. located on the surface of a cell, bacterium, virus particle, etc.

By “epitope” we mean an antibody attachment point on an antigen, i.e. an immunologically active binding site on an antigen to which an antibody or a B or T cell receptor can attach.

By “immunogen” we mean a substance that is able to provoke an adaptive immune response if injected on its own.

By “peptide”, “polypeptide” and “protein”, we mean a contiguous chain of amino acids linked by peptide bonds. Those of skill in the art will recognize that the term “peptide” is generally used for shorter amino acid chains, e.g. less than about 25 amino acids, whereas “polypeptide” generally refers to somewhat longer chains, e.g. about 25 to about 100 amino acids, and “proteins” are generally considered to be even larger, and may be several hundred or even a thousand or more amino acids in length. However, these lengths are not rigidly defined herein e.g. an amino acid chain with 115 amino acids or more may still be properly referred to as a “polypeptide”, particularly if the sequence thereof does not represent a distinct protein, as understood in the art. Likewise, in some contexts, a 30 amino acid chain may be properly referred to as a “peptide”. The terms “peptide” and “polypeptide” may be used interchangeably herein.

By a “neutralizing antibody” we mean an antibody that can neutralize (eliminate, decrease or attenuate) the ability of a pathogen to initiate and/or perpetuate an infection in a host. Without being bound by theory, it is believed that the neutralizing antibodies described herein do so by preventing (e.g. eliminating, or at least decreasing or attenuating) the ability of CMV virion particles to enter cells (e.g. epithelial, endothelial, or other cell types in which CMV relies on UL130 or UL131 for entry). In other words, the antibodies are capable of binding to CMV virions in a manner that prevents the CMV from entering and infecting the cells, when at least one of the neutralizing antibodies is bound to the CMV. Mucosal epithelial cells in particular are believed to be important for CMV transmission to naive hosts via the oral or sexual routes. Thus, by preventing infection of mucosal epithelial cells, neutralizing antibodies can provide sterilizing immunity by protecting the host from becoming infected by CMV. However, once CMV has gained entry via either oral or genital mucosal routes, the capacity to enter and replicate in epithelial, endothelial, or other cell types in which CMV relies on UL130 or UL131 for entry, is presumably important for CMV's ability to disseminate to the placenta during pregnancy, transmit to the fetus, and cause fetal disease, or, in immune compromised patients, to disseminate and cause end-organ disease (e.g., in liver, lung, kidney, eye, gastrointestinal tract, etc.). Thus, by preventing infection of tissue or circulating cells (e.g., epithelial, endothelial, or other cell types in which CMV relies on UL130 or UL131 for entry), neutralizing antibodies can provide therapeutic immunity by eliminating, decreasing, or attenuating viral dissemination to or subsequent replication and induction of damage at sites of disease. Those of skill in the art will recognize that neutralizing antibodies may completely prevent infection. Alternatively, much benefit accrues even if the efficiency of entry and infection is decreased by the antibodies, e.g. if the efficiency of entry into cells is decreased by at least about 25, 30, 35, 40, 45, 50, 55, 60, 65, 70, 75, 80, 85, 90, or 95% or greater, as determined by standardized tests that are known to those of skill in the art. As used herein, “efficiency” is defined as the number of cells infected in the presence of antibody as a percentage of the number infected in the absence of antibody.

### Combination Peptides, Polypeptides and Proteins

The peptides, polypeptides, and proteins of the invention are combination peptides, polypeptides, and proteins which include at least two components: 1) one or more copies of a peptide sequence that encompasses residues 27-46 of the CMV UL130 protein and/or one or more copies of a peptide sequence that encompasses residues 90-106 of the CMV UL131 protein (or functional variants of these two sequences); and 2) at least one other entity. Generally, the additional entity is proteinaceous in nature, and the combinations of the invention include, for example, chimeric and fusion constructs, as well as peptide-carrier complexes, etc.

In another embodiment, the invention provides combination peptides and polypeptides comprising multiple copies of one or the other or both of the UL130 and UL131 peptides. These peptides and polypeptides are of a length that is sufficient to render the construct antigenic, e.g. with at least about 2, 3, 4, 5, 6, 7, 8, 9, or 10 or more (e.g. about 15, 20, 25, 30, 35, 40, 45, or 50 or more total copies of peptides). Such constructs may be homogeneous with respect to the peptide subunits in the construct (i.e. only one type of peptide, either UL130 or UL131) is included. Alternatively, the construct may be mixed in that both UL130 and UL131 peptides are included. Further, functional variants of the UL130 and UL131 peptides (as described herein) may also be used to form the construct. The variants may be naturally occurring (e.g. isolated from different strains of CMV) or may be purposefully generated by introducing changes in a native or natural sequence, as is also described herein. Combination peptides and polypeptides of this type may also include, for example, linker or spacer sequences between the individual peptide units, or, alternatively, the peptide units may be directly linked (usually via a peptide bond) to each other with no intervening sequences.

### The UL130 and UL131 Peptide Component

The UL130 and UL131 peptides encompass epitopes which bind CMV-neutralizing antibodies. As shown in the Examples section below, immunogenic UL130-based peptide sequences vary somewhat from strain to strain. A consensus sequence developed from a comparison of the sequences from several different strains is as follows: X₁WX₂TLTANX₃NPSPPWSKLTY (SEQ ID NO: 7) wherein X₁═S or P; X₂═S or F; and X₃=Q or K. Herein, an exemplary peptide sequence derived from the TR strain is: SWSTLTANQNPSPPWSKLTY (UL130 residues 27-46; SEQ ID NO: 1). Other strains of CMV which were investigated had analogous sequences which differed slightly, namely exemplary sequences: PWSTLTANQNPSPPWSKLTY (SEQ ID NO: 2); PWFTLTANQNPSPPWSKLTY (SEQ ID NO:3); PWSTLTANKNPSPPWSKLTY (SEQ ID NO:4); and PWSTLTANQNPSPLWSKLTY (SEQ ID NO: 5). The immunogenic peptide sequence from UL131 did not display strain to strain variability in the strains that were examined (see Examples section below), and an exemplary sequence from those strains is as follows: SDFRRQNRRGGTNKRTT (UL131 residues 90-106; SEQ ID NO: 6).

An embodiment of the invention includes combination peptides, polypeptides and proteins which encompass or include one or more copies of the UL130 and/or UL131 peptides, or functional variants or derivatives of the UL130 and/or UL131 peptides. Functional variants are those which elicit the production of neutralizing antibodies as described herein, when administered to a mammalian subject or cell capable of producing antibodies. Such functional variants have at least about 50, 55, 60, 65, 70, 75, 80, 85, 90, 95, 96, 97, 98, 99% or more of the neutralizing activity of the peptides with sequences as set forth in SEQ ID NOS: 1-7, when measured using standard tests recognized by those of skill in the art, such as those described in the Examples section herein. Such variants or derivatives generally have at least about 50, 55, 60, 65, 70, 75, 80, 85, 90, 95, 95.5, 96, 96.5, 97, 97.5, 98, 98.5, 99, or 99.5% similarity or identity to UL130 and/or UL131 peptides found in nature, for example, to SEQ ID NOS: 1-7. As used herein, “% similarity” refers to the percentage of matching conservative changes and “% identity” refers to the percentage of matching of identical residues, when comparing SEQ ID NOS: 1-7 to a variant sequence, e.g. via alignment of the two sequences using a matrix, many of which are known to those of skill in the art. (This is sometimes also referred to as “homology”.) The “score” that is assigned to the variant or derivative (i.e. the fraction or percentage of identity) may be referred to as “% similarity” or “% identity”. Thus, a variant or derivative may be described as e.g. 99% identical or similar to e.g. SEQ ID NOS: 1-7. All such functional variants and derivatives are encompassed by the present invention.

In addition, identity and similarity may be determined using either the full length peptides as presented above, or foreshortened versions thereof. For example, from about 1 to about 10 (e.g. about 1, 2, 3, 4, 5, 6, 7, 8, 9, or 10, but preferably less than about 5) amino acids may be deleted (excluded) from the carboxy and/or amino termini of the peptides, and the resulting peptide is still encompassed by the present invention, so long as the resulting peptide remains functional. Further, from about 1-5 amino acids may be deleted or excluded from the sequence at any internal position (i.e. between any two non-contiguous amino acids) to form a functional variant or derivative, all of which are contemplated by the invention.

In addition, variants of the UL130 and UL131 peptides include shorter functional epitopes located within these sequences, comprised of e.g. at least 6 contiguous amino acids. For UL130, such shorter sequences may be about 6, 7, 8, 9, 10, 11, 12, 13, 14, 15, 16, 17, 18, or 19 contiguous amino acids located at (i.e. beginning and ending at) any position within the 20 amino acid sequence. For UL131, 6, 7, 8, 9, 10, 11, 12, 13, 14, 15, or 16 contiguous amino acids located at (i.e. beginning and ending at) any position within the 17 amino acid sequence.

Functional variants also include peptides which have changes or mutations (e.g., at least about one, two, or four, and/or generally less than 15, 10, 5, or 3) relative to the sequences described herein (e.g., conservative or non-essential amino acid substitutions), which do not have a substantial effect on peptide function. Whether or not a particular substitution will be tolerated, i.e., will not adversely affect biological properties, can be predicted, e.g., by evaluating whether the mutation is conservative or by the method of Bowie, et al. (1990) Science 247:1306-1310.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Peptides encompassed by the invention may include one or more conservative substitutions.

In addition, certain chemical or other modifications of the peptides may be tolerated without significantly compromising the ability of the peptides to elicit the production of neutralizing antibodies. Such modifications include, for example, the addition of sequences which aid in synthesis and/or isolation of the peptides is encompassed (e.g. histidine or other tags, etc.). In addition, the sequences may be modified to impart stability, e.g. by preventing protease digestion by pegylation; or modified e.g. by amidation or esterification of the C-terminus; acetylation of the amino terminus; phosphorylation, methylation, sulfation, or modified by the inclusion of signal or targeting sequences; or to facilitate chemical coupling (e.g., addition of a C-terminal cycteine to facilitate coupling to a maleiamide-activated carrier) or binding (e.g. addition of a C-terminal biotin to allow binding to streptavidin) to carriers, etc. All such modifications are encompassed by the present invention.

### The Additional Component

In one embodiment, the combination peptides, polypeptides and proteins of the invention also include one or more additional entities (components). The second component (and/or the third, forth, fifth, etc. components, if more than one additional component is present) is generally, although not necessarily always, proteinaceous in nature, i.e. is usually another amino acid sequence which differs from the UL130 and UL131 peptides. In some embodiments, the second entity is heterologous, i.e. is derived or taken from a non-CMV source, e.g. from another species. However, in other embodiments, the additional entity may be derived from CMV, and may be, for example, another CMV protein (e.g. gH, gB, full length UL130 or UL131, etc.). According to the invention, one or more copies of a UL 130 and/or a UL131 peptide is/are associated with, e.g. embedded, incorporated, fused, attached, conjugated, linked to, etc. the additional entity. Generally, the attachment or association is covalent, although this need not always be the case; other types of chemical coupling are also encompassed. The UL130 and/or UL131 peptides may be attached to the additional component(s) by any technique known to those of skill in the art, including via production of a genetically engineered, recombinant nucleotide sequence that can be translated into the desired combination peptide, polypeptide or protein sequence, or via chemical coupling of two or more components of interest which are to be included in the combination peptide, polypeptide or protein, or via non-covalent binding such as mediated by biotin/streptavidin.

In some embodiments, the additional entity renders the UL130 and 131 peptide component(s) suitable for administration to human subjects. For example, in one embodiment, the additional component is a pharmaceutically acceptable carrier protein, especially an immunogenic carrier protein. Exemplary carrier proteins include but are not limited to: albumin, ovalbumin, Pseudomonas exotoxin, tetanus toxin, ricin toxin, diphtheria toxin, cholera toxin, heat labile enterotoxin, meningococcal protein, keyhole lympet hemocyanin, epidermal growth factor, fibroblast growth factor, transferrin, platelet-derived growth factor, poly-L-lysine, poly-L-glutamine, mannose-6-phosphate, various cell surface and membrane proteins, various polyepitope carriers e.g. comprising CD4+ cell epitopes as described in U.S. Pat. No. 7,538,207 (the complete contents of which is hereby incorporated by reference), and the like. In addition, the carrier or the carrier plus peptide(s) complex may also comprise one or more conjugated polysaccharides, (e.g. bacterial polysaccharides such as thymus independent (TI) polysaccharide), and others.

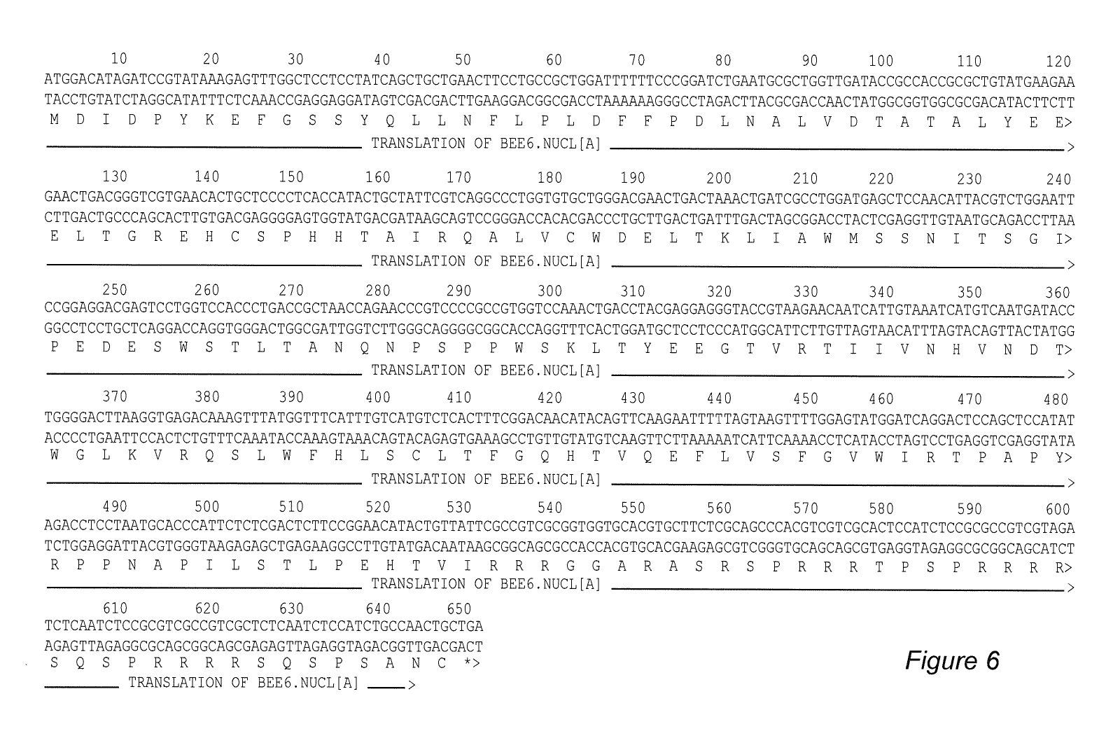

In another embodiment, the combination peptides, polypeptides or proteins are chimeric or fusion constructs. Such constructs contain one or more of the UL130 and/or UL131 peptides (or functional variants thereof) plus an additional entity, (e.g. other sequences which differ from the UL130 and UL131 peptides) in a single contiguous polypeptide chain. However, such constructs are not full length UL130 or full length UL131. In this embodiment, the “other sequences” constitute the “additional entity” as described above. The chimeras/fusions may include one or multiple copies of one or both of the UL130 and UL131 peptides. As the additional entity, chimeras/fusions may include, for example, linker or spacer sequences between the peptide sequences of interest (i.e. the reactive peptides which contain epitopes to which it is desired to elicit an immune response), and/or other sequences which, for example, act as adjuvants to stimulate antigenicity, which confer stability to the amino acid chain, targeting sequences, antibodies or portions of antibodies, proteins known to be antigenic (e.g. a hepatitis B core protein), etc. Such constructs may be produced either recombinantly (by organisms that are genetically engineered to contain and express nucleic acid sequences that encode the construct, e.g. E. coli, baculovirus, adenovirus, etc.) or by chemical synthesis, as is well known in the art. In one embodiment, one or more UL 130 and/or UL131 peptides is genetically engineered into a hepatitis virus B core antigen (HBcAg) protein to form a chimeric or fusion polypeptide/protein as described in Example 2 below. An exemplary HBcAg chimeric sequence is shown in FIG. 6.

### Nucleic Acids

The invention also encompasses nucleic acid sequences which encode each of the combination peptide, polypeptide and protein species described herein. The nucleic acids may be, for example, DNA or RNA, and may be single or double stranded, and may also be contained within a larger nucleic acid sequence that forms, for example, a vector for expression of the peptide(s)/polypeptide(s)/protein(s) or a shuttle vector. Exemplary vectors include but are not limited to plasmids, cosmids, various viral vectors and modified viral genomes which are known in the art for use in expressing peptides and polypeptides recombinantly (e.g. adenoviral vectors), as well as bacterial and insect vectors. Vectors are described in more detail below.

Those of skill in the art will recognize that, due to the redundancy of the genetic code, many different nucleic acid sequences exist or can be developed which would encode any one of the specific peptides/polypeptides described herein, and all such sequences are encompassed by the invention. Further, codon optimized version of nucleic acids are also contemplated.

### Antibodies

The peptide, polypeptide and protein sequences disclosed herein can be used as immunogens to generate antibodies (e.g. IgM, IgG, etc.) using standard techniques for polyclonal and monoclonal antibody preparation. The invention thus encompasses both antibodies specific for the constructs described herein, and methods of making antibodies to the constructs. By “specific for the constructs described herein” we mean that the antibodies react specifically with the peptide, polypeptide and protein species described herein, but not with other amino acid sequences.

Antibodies are typically prepared by administering one or more of the combination peptides/polypeptides/proteins of the invention (which have been substantially purified) to a suitable subject, (e.g., rabbit, goat, mouse or other mammal) with or without other adjuvants or immunogens. An appropriate immunogenic preparation can contain, for example, recombinantly expressed or chemically synthesized peptides/polypeptides/proteins. The preparation can further include one or more adjuvants, such as Freund's complete or incomplete adjuvant, various non-organic adjuvants such as aluminum salts (aluminum phosphate and aluminum hydroxide), alum, etc.; various organic adjuvants e.g. squalene and other oil-based adjuvants; virosomes; MF59; QS21 (a purified plant extract derived from the Soap bark tree (Quillaja saponaria). The extract contains water soluble triterpene glucoside compounds, which are members of a family of plant-based compounds called saponins); immunostimulatory oligonucleotides, e.g. those having at least one CpG dinucleotide; or similar immunostimulatory agents. Immunization of a suitable subject with such a preparation induces a polyclonal antibody response.

Accordingly, another aspect of the invention pertains to antibodies that react specifically with any of the combination peptides/polypeptides/proteins described herein, or functional variants thereof. The term “antibody” as used herein refers to immunoglobulin molecules and immunologically active portions of immunoglobulin molecules i.e., molecules that contain an antigen binding site which specifically binds (immunoreacts with) at least one antigen or epitope. Examples of immunologically active portions of immunoglobulin molecules include F(ab) and F(ab′)₂ fragments which can be generated by treating the antibody with an enzyme such as pepsin. The invention provides polyclonal and monoclonal antibodies. The term “monoclonal antibody” or “monoclonal antibody composition”, as used herein, refers to a population of antibody molecules that contain only one species of an antigen binding site capable of immunoreacting with a particular epitope. A monoclonal antibody composition thus typically displays a single binding affinity for a particular epitope with which it immunoreacts.

Polyclonal antibodies can be prepared as described above by immunizing a suitable subject with an immunogen such as the combination peptides/polypeptides/proteins described herein, or functional variants thereof. The antibody titer in the immunized subject can be monitored over time by standard techniques, such as with an enzyme linked immunosorbent assay (ELISA) using e.g. immobilized peptides. If desired, the antibody molecules can be isolated from the mammal (e.g., from the blood) and further purified by well known techniques, such as protein A chromatography to obtain the IgG fraction. At an appropriate time after immunization, e.g., when the antibody titers are highest, antibody-producing cells can be obtained from the subject and used to prepare monoclonal antibodies by standard techniques, such as the hybridoma technique originally described by Kohler and Milstein (1975) Nature 256:495-497) (see also, Brown et al. (1981) J Immunol. 127:539-46; Brown et al. (1980) J Biol. Chem. 255:4980-83; Yeh et al. (1976) PNAS 76:2927-31; and Yeh et al. (1982) Int. J. Cancer 29:269-75), the more recent human B cell hybridoma technique (Kozbor et al. (1983) Immunol Today 4:72), the EBV-hybridoma technique (Cole et al. (1985), Monoclonal Antibodies and Cancer Therapy, Alan R. Liss, Inc., pp. 77-96) or trioma techniques. The technology for producing monoclonal antibody hybridomas is well known. Briefly, an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds the immunogen. Any of the many well known protocols used for fusing lymphocytes and immortalized cell lines can be applied for the purpose of generating an monoclonal antibody (see, e.g., G. Galfre et al. (1977) Nature 266:55052; Gefter et al. Somatic Cell Genet., cited supra; Lerner, Yale J Biol. Med., cited supra; Kenneth, Monoclonal Antibodies, cited supra). Moreover, the ordinarily skilled worker will appreciate that there are many variations of such methods which also would be useful. Typically, the immortal cell line (e.g., a myeloma cell line) is derived from the same mammalian species as the lymphocytes. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies e.g., using a standard ELISA assay. The invention also encompasses immortal cell lines which express the antibodies of the invention.

Alternative to preparing monoclonal antibody-secreting hybridomas, a monoclonal antibody can be identified and isolated by screening a recombinant combinatorial immunoglobulin library (e.g., an antibody phage display library), or by screening human B cell populations from CMV-infected or immunized human subjects to identify B cell clones that secrete antibodies reactive to the desired epitopes.

Additionally, recombinant antibodies, such as chimeric and humanized monoclonal antibodies, comprising both human and non-human portions, which can be made using standard recombinant DNA techniques, are within the scope of the invention.

### Immunogenic Compositions and Vaccines

### Peptide, Polypeptide and Protein Vaccines

The present invention provides compositions for use in eliciting an immune response in and/or vaccinating an individual against CMV, especially against infection of epithelial cells (e.g. oral and vaginal mucosal epithelial cells) by CMV. The compositions include one or more isolated and substantially purified combination peptides, polypeptides or proteins as described herein, and a pharmacologically or physiologically suitable (compatible) carrier that is suitable for administration to a human. By “isolated and substantially purified” we mean that the peptide, polypeptide, or protein is substantially (e.g. at least about 75, 80, 85, 90, 95% or more) purified, i.e. free from other chemical substances (e.g. other molecules, cellular components or debris, chemical reagents, etc.) other than those which are purposefully added to the composition. Isolation and purification of peptides, polypeptides, or proteins is well know in the art and may involve the use of various types of techniques which separate a peptide of interest from undesirable or unwanted material, including but not limited to chromatography, gels, precipitation techniques, etc.

The preparation of compositions for use as vaccines and/or to elicit an immune response is well known to those of skill in the art. Typically, such compositions are prepared either as liquid solutions or suspensions, however solid forms such as tablets, pills, powders and the like are also contemplated. Solid forms suitable for solution in, or suspension in, liquids prior to administration may also be prepared. The preparation may also be emulsified. The active ingredients may be mixed with excipients which are pharmaceutically acceptable and compatible with the active ingredients. Suitable excipients are, for example, water, saline, dextrose, glycerol, ethanol and the like, or combinations thereof. In addition, the composition may contain minor amounts of auxiliary substances such as wetting or emulsifying agents, pH buffering agents, and the like. In addition, the composition may contain adjuvants. If it is desired to administer an oral form of the composition, various thickeners, flavorings, diluents, emulsifiers, dispersing aids or binders and the like may be added. The composition of the present invention may contain any such additional ingredients so as to provide the composition in a form suitable for administration. The final amount of peptide or encoding nucleic acid in the formulations may vary. However, in general, the amount in the formulations will be from about 1-99%. See, for example, Remingion's Pharmaceutical Sciences 16th edition, Osol, A. Ed. (1980).

In one embodiment, the invention provides immunogenic compositions which comprise the combination peptides, polypeptides, or proteins disclosed herein, in combination with one or more other anti-CMV immunogenic entities, i.e. the sequences of the invention may be part of a multivalent vaccine or immunostimulatory composition. Exemplary entities for such combinations include but are not limited to one or more of: other CMV antigens, examples of which include but are not limited to: antigens from other proteins in the gH/gL/UL128-131 complex, the gB protein or modified but antigenic versions thereof (e.g. genetically modified versions containing surface exposed residues, or versions from which transmembrane segments have been removed, etc.) glycoprotein 0 of CMV; glycoprotein M, glycoprotein N, pp 65, etc.

### Nucleic Acid Vaccines

The invention also contemplates immunostimulatory compositions and vaccine preparations that include nucleic acids encoding the combination peptides, polypeptide or proteins described herein, as well as nucleic acids that encode the UL130 and UL131 peptides, with or without an additional component. Typically, the nucleic acid is DNA or RNA housed in a vector suitable for use in nucleic acid-based vaccines. Exemplary vectors include but are not limited to various recombinant viral and bacterial expression vectors (in which the nucleic acid is associated with a suitable promoter to drive expression) such as adenoviral vectors, mycobacterial vectors, pox-virus vectors, recombinant alpha-virus based vectors, and others known to those of skill in the art. (For example see issued U.S. Pat. Nos. 8,012,747 and 7,998,733, the complete contents of which are hereby incorporated by reference.) Alternatively, “naked” DNA or RNA may be administered, e.g. in a plasmid or other suitable vector that is not viral or bacterial. In this case, the encoding nucleic acid is also generally associated with a promoter that drives expression of the peptide, polypeptide or protein after administration. Vectors are described in more detail below.

Nucleic acid vaccine compositions are typically made as described above for peptide/polypeptide/protein vaccines, i.e. in a formulation with a physiologically compatible carrier suitable for use in humans, with appropriate additives and/or excipients, etc. Routes of administration may also be similar, except that if “naked” DNA or RNA is administered, this is usually by way of e.g. a gene gun, attachment of the nucleic acid to gold beads, via liposomes, etc. Naked nucleic acid vaccines may also include compounds or materials intended as adjuvants, such as poloxamers or cationic lipids (e.g., Vaxfectin®).

### Antibody Compositions

Similarly, the invention provides compositions comprising one or more antibodies as described herein. Such compositions are generally used to administer the antibodies e.g. as therapeutic agents to subjects in need thereof, in order to prevent, attenuate or treat CMV infections. The preparation and administration of antibody compositions is generally the same as described for vaccine administration above, e.g. the antibodies are substantially purified, are usually administered with a physiologically compatible carrier, with the amount of antibody in the compositions ranging from about 1 to 99% of the composition, etc. Vector” includes shuttle and expression vectors. Typically, the plasmid construct will also include an origin of replication (e.g., the ColE1 origin of replication) and a selectable marker (e.g., ampicillin or tetracycline resistance), for replication and selection, respectively, of the plasmids in bacteria. An “expression vector” refers to a vector that contains the necessary control sequences or regulatory elements for expression of the antibodies including antibody fragment of the invention, in bacterial or eukaryotic cells. Suitable vectors are disclosed below.

### Vectors, Host Cells and Recombinant Methods

The invention provides vectors and host cells including a nucleic acid of the present invention, as well as recombinant techniques for the production of combination peptides, polypeptides and proteins of the present invention. Vectors of the invention include those capable of replication in any type of cell or organism, including, e.g., plasmids, phage, cosmids, and mini chromosomes, etc. In various embodiments, vectors including a polynucleotide of the present invention are vectors suitable for propagation or replication of the polynucleotide, or vectors suitable for expressing a polypeptide of the present invention. Such vectors are known in the art and commercially available. Expression may be transient or stable, with stable generally being preferred for polypeptide production, whereas transient expression may be preferred in nucleic acid vaccine vectors.

Polynucleotides of the present invention are synthesized, whole or in parts that are then combined, and inserted into a vector using routine molecular and cell biology techniques, including, e.g., subcloning the polynucleotide into a linearized vector using appropriate restriction sites and restriction enzymes. Polynucleotides of the present invention may be amplified by polymerase chain reaction using oligonucleotide primers complementary to each strand of the polynucleotide. These primers may also include restriction enzyme cleavage sites to facilitate subcloning into a vector. The replicable vector components generally include, but are not limited to, one or more of the following: a signal sequence, an origin of replication, and one or more marker or selectable genes.

In order to express a combination peptide, polypeptide or protein of the present invention, the encoding nucleotide sequences, or functional equivalents thereof, are inserted into an appropriate expression vector, i.e., a vector that contains the necessary elements for the transcription and translation of the inserted coding sequence. Methods well known to those skilled in the art are used to construct expression vectors containing sequences encoding a polypeptide of interest and appropriate transcriptional and translational control elements. These methods include in vitro recombinant DNA techniques, synthetic techniques, and in vivo genetic recombination. Such techniques are described, for example, in Sambrook, J., et al. (2001) Molecular Cloning, A Laboratory Manual, Cold Spring Harbor Press, Plainview, N.Y., and Ausubel, F. M. et al. (1989) Current Protocols in Molecular Biology, John Wiley & Sons, New York. N.Y.

A variety of expression vector/host systems are utilized to contain and express polynucleotide sequences. These include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus, CaMV; tobacco mosaic virus, TMV) or with bacterial expression vectors (e.g., Ti or pBR322 plasmids); or animal cell systems.

The “control elements” or “regulatory sequences” present in an expression vector are those non-translated regions of the vector, e.g., enhancers, promoters, 5′ and 3′ untranslated regions, that interact with host cellular proteins to carry out transcription and translation. These elements may vary in their strength and specificity. Depending on the vector system and host utilized, any number of suitable transcription and translation elements, including constitutive and inducible promoters, are used.

Examples of promoters suitable for use with prokaryotic hosts include the phoa promoter, β.-lactamase and lactose promoter systems, alkaline phosphatase promoter, a tryptophan (trp) promoter system, and hybrid promoters such as the tac promoter. However, other known bacterial promoters are suitable. Promoters for use in bacterial systems also usually contain a Shine-Dalgarno sequence operably linked to the DNA encoding the polypeptide. Inducible promoters such as the hybrid lacZ promoter of the pBLUESCRIPT phagemid (Stratagene, La Jolla, Calif.) or pSPORT1 plasmid (Gibco BRL, Gaithersburg, Md.) and the like may be used.

In mammalian cell systems, promoters from mammalian genes or from mammalian viruses are generally preferred. Polypeptide expression from vectors in mammalian host cells may be controlled, for example, by promoters obtained from the genomes of viruses such as polyoma virus, fowlpox virus, adenovirus (e.g., Adenovirus 2), bovine papilloma virus, avian sarcoma virus, cytomegalovirus (CMV), a retrovirus, hepatitis-B virus, Simian Virus 40 (SV40), etc.; and heterologous mammalian promoters, e.g., the actin promoter or an immunoglobulin promoter, and from heat-shock promoters, provided such promoters are compatible with the host cell systems. If it is necessary to generate a cell line that contains multiple copies of a sequence, vectors based on SV40 or EBV may be advantageously used with an appropriate selectable marker. One example of a suitable expression vector is pcDNA3.1 (Invitrogen, Carlsbad, Calif.), which includes a CMV promoter.

A number of viral-based expression systems are available for mammalian expression of polypeptides. For example, in cases where an adenovirus is used as an expression vector, sequences encoding a polypeptide of interest are ligated into an adenovirus transcription/translation complex consisting of the late promoter and tripartite leader sequence. Insertion in a non-essential E1 or E3 region of the viral genome is used to obtain a viable virus that is capable of expressing the polypeptide in infected host cells (Logan, J. and Shenk, T. (1984) Proc. Natl. Acad. Sci. 81:3655-3659). In addition, transcription enhancers, such as the Rous sarcoma virus (RSV) enhancer, are used to increase expression in mammalian host cells.

In bacterial systems, any of a number of expression vectors are selected depending upon the use intended for the expressed polypeptide. For example, when large quantities are desired, vectors that direct high level expression of fusion proteins that are readily purified are used. Such vectors include, but are not limited to, the multifunctional E. coli cloning and expression vectors such pET (Stratagene), in which the sequence encoding the polypeptide of interest is ligated into the vector in frame with sequences for the amino-terminal Met and the subsequent 7 residues of .beta.-galactosidase, so that a hybrid protein is produced; pIN vectors (Van Heeke, G. and S. M. Schuster (1989) J. Biol. Chem. 264:5503-5509); and the like. pGEX Vectors (Promega, Madison, Wis.) are also used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. Proteins made in such systems are designed to include heparin, thrombin, or factor Xa protease cleavage sites so that the cloned polypeptide of interest can be released from the GST moiety at will.

Similarly, those of skill in the art are aware of expression systems for yeast, plant and insect systems. For example, see U.S. Pat. No. 7,982,012, the complete contents of which is hereby incorporated by reference. Further, as will be understood by those of skill in the art, for any expression system, other sequences which function to facilitate or enable accurate and robust translation may also be included, e.g. enhancer sequences, specific initiation signals, sequences necessary for the termination of transcription and for stabilizing the mRNA, stop signals, etc.

Suitable host cells for cloning or expressing the DNA in the vectors herein are the prokaryote, yeast, plant or higher eukaryote cells described above, and as known in the art. Host cells may be transformed with the above-described expression or cloning vectors for polypeptide production and cultured in conventional nutrient media modified as appropriate for inducing promoters, selecting transformants, or amplifying the genes encoding the desired sequences.

### Methods

### Methods of Vaccination and of Eliciting an Immune Response (Such as the Generation of Antibodies)

The invention also encompasses methods of treating and/or preventing CMV infection and transmission in subjects in need thereof, methods of eliciting an immune response to CMV in subjects in need thereof, and methods of vaccinating an individual against CMV infection. The methods involve administering a vaccine or immune response eliciting (stimulating) composition of the invention by any of the many suitable means which are well known to those of skill in the art, including but not limited to: by injection (e.g. intravenous, intraperitoneal, intramuscular, subcutaneous, and the like), by inhalation, orally, intravaginally, intranasally, by ingestion of a food or probiotic product containing the peptide(s), as eye drops, via sprays, by absorption through epithelial or mucocutaneous linings (e.g., nasal, oral, vaginal, rectal, gastrointestinal mucosa, and the like), etc. In preferred embodiments, the mode of administration is by injection. In addition, the compositions may be administered in conjunction with other treatment modalities such as substances that boost the immune system, various chemotherapeutic agents, adjuvants, other antigens, etc. In one embodiment, the proprietary adjuvant MF59 is utilized. MF59™ adjuvant (MF59TMC.1) is an oil-in-water emulsion (o/w) consisting of small (˜160 nm in diameter), uniform, and stable microvesicles, consisting of a drop of oil surrounded by a monolayer of non-ionic detergents. The oil is squalene, which is obtained from shark liver. Squalene is a natural component of cell membranes; it is found in human sebum (a skin surface lipid) and is a naturally occurring hydrocarbon precursor of cholesterol. Squalene droplets are stabilized by addition of 2 non-ionic surfactants, a low hydrophilic-lipophilic balance (HLB) surfactant, Polysorbate 80 (Tween 80), which is widely used as an emulsifier in foods, cosmetics and pharmaceuticals, including parenteral formulations [12], and sorbitan triolate (common name is Span 85), as described by Schultze et. al. (Vaccine 26 (2008) 3209-3222), the entire contents of which is hereby incorporated by reference. Other adjuvants that may be administered include those listed above in the section entitled “Antibodies”.

The amount of immunogenic combination peptide, polypeptide, or protein that is administered to an individual will vary from case to case, and is best determined by a skilled medical practitioner (e.g. a physician, nurse practitioner, etc.) using guidelines established e.g. during clinical trials. However, the dosage range is typically from about 1 to about 1000 mg/kg of total body weight, or from about 5 to about 500 mg/kg of total body weight. This amount may vary based on, e.g. the route of administration, the age, gender, overall health, and other characteristics of the recipient who is receiving the composition.

In some embodiments of the invention, the compositions of the invention are administered to a subject who is at risk of or likely to experience CMV exposure, or who is known or likely to have been or exposed, but has not yet developed a CMV infection. However, in other embodiments, the composition is administered to individuals who have already developed an infection, in order to curtail the extent of infection in the individual and hasten recovery, and/or to prevent transmission to others. Subjects to whom the compositions are administered are generally humans.

Target populations for vaccination with a CMV vaccine include but are not limited to young children (even infants and babies), preadolescent girls (similar to HPV vaccine), women of child bearing age, and any subject scheduled to undergo tissue or bone marrow transplantation, due to the likelihood of administration of immune suppressants.

The protocol for subjects to whom the compositions are administered typically follows the guidelines for other vaccines, e.g. childhood vaccination at from 1-3 months, followed by booster doses at 3-6 months, and/or at 6 months to 1 year, and possibly yearly thereafter, or every 5 years or every 10 years, as needed to maintain immunity. The schedule for adults may be similar, or may be less frequent in individuals with mature immune systems, e.g. one administration, followed by a yearly booster, or a booster after 5 or 10 years, as needed. Those of skill in the art will recognize that most protein/peptide-based vaccines are likely to require at least two initial doses, perhaps followed by a third dose at 6-12 months. Those of skill in the art will recognize that adjustments to the protocol may be made to account for a subject individual health status, and/or based on cumulative results obtained from tracking other subjects either during clinical trials, or in a non-experimental clinical setting.

### Methods of Administering Antibodies

The invention also provides methods of administering antibody compositions as described herein. The target populations are similar to those to whom a vaccine may be administered, except that the effect of the antibodies may be more immediate, and may be more suitable for those who are immune compromised and would be unlikely to mount a robust immune response to CMV antigens. Such individuals include, for example, individuals undergoing immune suppression therapy, AIDS patients, and individuals with other conditions whose immune systems may be compromised, e.g. the very young or the elderly, those with chronic illnesses or who are undergoing e.g. chemotherapy, etc. However, those of skill in the art will recognize that antibodies may be administered to any subject who may derive benefit therefrom.

### Diagnostics and Research Tools

The combination peptides/polypeptides/proteins peptides, and functional variants thereof, the nucleic acids, and the antibodies described herein have a plethora of diagnostic and research investigational applications, as those of skill in the art will recognize. For example, the peptides, polypeptides, and proteins may be used as screening tools to isolate monoclonal antibodies or cDNA sequences of monoclonals that bind to the peptides. In particular, the amino acid based entities may be used to generate antibodies; to perform antibody titer testing in a diagnostic test (e.g. to determine whether a subject has been exposed to CMV or has a CMV infection; as a prognostic indicator for protection from infection (e.g. congenital infection); as a prognostic indicator for severity of CMV disease in immune compromised patients or fetal disease in congenital infection, etc.

The nucleic acids may be used e.g. to diagnose CMV in a patient in need thereof, to genotype CMV viruses, or to determine potential for reinfection by CMV strains that differ antigenically within these peptide sequences, etc.

The antibodies may be used for diagnostic purposes (e.g. to diagnose CMV in a patient by detecting the presence of CMV related or derived proteins and peptides in tissue or bodily fluid samples, mucous etc.) or as therapeutic agents, either prophylactically (passive immunization) or therapeutically (to prevent or reduce disease from an established infection, etc.

The invention provides kits comprising one or more of the combination peptides/polypeptides/proteins peptides, nucleic acids, and antibodies described herein, e.g. for use as diagnostic assays in clinical settings, or for use as research tools in laboratory settings.

For such applications, the combination peptides, polypeptides, proteins; nucleic acids; and antibodies of the invention may be conjugated to substrates such as e.g. beads, or immobilized e.g. in multi-well plates or test tubes, etc., and may be used together with other indicator substances such as various fluorescent or radioactive labels, as will be understood by those of skill in the art.

### EXAMPLES

### Example 1

### Peptides from Cytomegalovirus UL130 and UL131 Proteins Induce High Titer Antibodies that Block Viral Entry into Mucosal Epithelial Cells

Cytomegalovirus infections are an important cause of disease for which no licensed vaccine exists. Recent studies have focused on the gH/gL/UL128-131 complex as antibodies to gH/gL/UL128-131 neutralize viral entry into epithelial cells. Prior studies have used cells from the retinal pigment epithelium, while to prevent transmission, vaccine-induced antibodies may need to block viral infection of epithelial cells of the oral or genital mucosa. We found that gH/gL/UL128-131 is necessary for efficient viral entry into epithelial cells derived from oral and genital mucosa, that short peptides from UL130 and UL131 elicit high titer neutralizing antibodies in rabbits, and that such antibodies neutralize viral entry into epithelial cells derived from these relevant tissues. These results suggest that single subunits or peptides may be sufficient to elicit potent epithelial entry neutralizing responses and that secretory antibodies to such neutralizing epitopes have the potential to provide sterilizing immunity by blocking initial mucosal infection.

### 1. Introduction

Two questions relevant to the design of effective vaccine strategies are evaluated herein. First, do antibodies targeting gH/gL/UL128-131 complex neutralize viral entry into tissues relevant to vaccine protection? For example, mucosal and secretory antibodies that neutralize viral entry into epithelial cells of the oral or genital epithelium may prevent or reduce viral transmission. Second, will individual subunits suffice or will more than one subunit, perhaps the entire complex, be required for a vaccine? We observed that a functional gH/gL/UL128-131 complex is essential for efficient CMV entry into epithelial cells derived from both airway and genital mucosa, demonstrated that immunization of rabbits with short peptides derived from UL130 or UL131 is sufficient to achieve high epithelial entry neutralizing titers, and showed that these sera are effective at blocking CMV entry into mucosal epithelial cells. Our results indicate that subunit or peptide immunogens will elicit epithelial entry neutralizing responses and that successful active immunization may provide sterilizing immunity.

### 2. Materials and Methods

### 2.1 Viruses

Virus HB15-t178b was derived from bacterial artificial chromosome (BAC) clone HB15Tn7Δk [13], which contains the CMV strain AD169 genome [14], by transposition of a green fluorescent protein (GFP) reporter cassette into the attTn7 site, as described [15]. Virus HB15-t178b retains a UL131 frame shift mutation intrinsic to strain AD169. Virus BADrUL131-Y4 was derived from a different BAC clone of the CMV strain AD169 genome [16] that was first modified to express GFP [17] and then, by repair of the UL131 mutation, to express a functional UL131 protein [18]. Viral stocks were prepared from cell culture media that was clarified by centrifugation, adjusted to 0.2 M sucrose, aliquoted, stored at −80° C., and titered on MRC-5 cells by limiting-dilution in 96-well plates as described [19].

### 2.2 Cells

Table 1 summarizes the cell lines used. MRC-5 (ATCC CCL-171), ARPE-19 (ATCC CRL-2302), and HBE4-E6/E7 (ATCC CRL-2078) cells were obtained from ATCC. HFK-2, Cx, V428, and HTE 21505 were derived and immortalized by retroviral transduction of human papilloma virus-16 E6E7 as previously described [20]. MRC-5 and ARPE-19 cells were propagated in high glucose Dulbecco's modified Eagle medium (Gibco-BRL) supplemented with 10% fetal calf serum (HyClone Laboratories), 10,000 FU/L penicillin, 10 mg/L streptomycin (Gibco-BRL) (DMEM). HFK-2, Cx, V428, and HTE 21505 cells were propagated in keratinocyte serum free medium (KSFM, GIBCO 17005042) supplemented with 5 ng/mL human recombinant epidermal growth factor 1-53 (Invitrogen) and 0.05 mg/mL bovine pituitary extract (Invitrogen). HBE4-E6/E7 cells were propagated with KSFM supplemented with 5 ng/ml human recombinant epidermal growth factor 1-53, 0.05 mg/ml bovine pituitary extract, and 10 ng/ml cholera toxin (Sigma). All cell cultures were maintained at 37° C. in a 5% CO₂ atmosphere.

TABLE 1
Cell lines

| | |
|---|---|
| Cell line | Tissue (cell type) |

fibroblast

| | |
|---|---|
| MRC-5 | fetal lung |

epithelial

| | |
|---|---|
| ARPE-19 | retinal pigment epithelium |
| HFK-2 | foreskin (keratinocyte) |
| Cx | cervix (keratinocyte) |
| V428 | vagina |
| HTE 21505 | tonsil |
| HBE4-E6/E7 | bronchus |

### 2.3 Entry Assay

Virus stocks were carefully titered using MCR-5 fibroblast cells, then matching amounts of HB15-t178b and BADrUL131-Y4 were used to infect replicate cultures of confluent cells prepared in 24-well plates. After 24 h the cultures were washed three times with PBS and fresh medium was added. Photomicrographs were taken daily post infection using an Olympus LX70 Inverted UV microscope.

### 2.4 Rabbit Immunizations

Antisera were initially produced for the purpose of antigen detection and immunoprecipitation. The amino acid sequence of each protein was evaluated using computer algorithms that predict hydrophilic, antigenic, and surface exposed domains. From these results one peptide from each protein was selected based on empirical experience that N- or C-terminal positions, charged residues, and prolines are desirable. Peptides DQYLESVKKIHKRLDV (UL128 residues 147-162; SEQ ID NO: 3), SWSTLTANQNPSPPWSKLTY (UL130 residues 27-46; SEQ ID NO: 1), and SDFRRQNRRGGTNKRTT (UL131 residues 90-106; SEQ ID NO: 2) were synthesized with C-terminal cysteines by PeptidoGenics (Berkley, Calif.) and coupled to maleimide activated keyhole limpet hemocyanin (KLH) under conditions that produce conjugates in which the peptides comprise 15-30% of the mass. For each peptide one New Zealand White rabbit was immunized with 500 to 1000 μg of KLH-conjugated peptide mixed with Freund's adjuvant, then boosted three times at 4-6 week intervals with decreasing doses of KLH-conjugated peptides (250 μg, 100 μg, and 50 μg) in TiterMax Gold adjuvant (Sigma, St. Louis, Mo.). An isoleucine at position 10 of the UL128 peptide was unintentionally inserted. However, this does not prevent recognition of native UL128 (which lacks the isoleucine) by the UL128 antiserum. Indeed, all three antisera have been extensively characterized elsewhere and shown to react specifically with UL128, UL130, or UL131 by immunoprecipitation and immunoblotting [21, 22].

### 2.5 Neutralization Assays

Neutralizing activities were determined by preparing 1:10 dilutions of each serum followed by additional 2-fold serial dilutions in ARPE-19 culture medium. Each dilution was mixed with an equal volume of ARPE-19 culture medium containing 500 pfu of BADrUL131-Y4, incubated for 1 h at 37° C., then added to the wells of 384-well plates containing confluent ARPE-19 monolayers. Each serum was assayed in triplicate and representative photomicrographs were taken using a Nikon Eclipse TS100 inverted UV microscope at four days post infection. GFP fluorescence was measured seven days post infection using a PerkinElmer Victor3 V 1420 Multilable Counter. Fifty percent inhibitory concentration (IC₅₀) values and standard errors of the means were calculated using Prism software (GraphPad Software, Inc.) by plotting the means of triplicate GFP values for each serum dilution against log₂ serum concentration, calculating the best fit four-parameter equation for the data, and interpolating the serum dilution at the mid-point of the curve as the IC₅₀ neutralizing titer. To evaluate neutralization of viral entry into mucosal epithelial cells rabbit anti-peptide sera were used at a 1:20 dilution and photomicrographs were taken seven days post infection.

### 3. Results

3.1 A Functional gH/gL/UL128-131 Complex is Required for Efficient CMV Entry into Epithelial Cells from Mucosal Tissues

To determine the role of gH/gL/UL128-131 in CMV entry into epithelial cells from mucosal tissues, we compared the entry efficiencies of two GFP-tagged viruses (one expressing and one lacking the gH/gL/UL128-131 complex) by measuring the number of GFP+ cells observed at different times after infection. Strain AD169 is the standard laboratory/reference strain of CMV. It has a frame shift mutation in the UL131 gene that disrupts expression of the UL131 protein [9] and prevents formation and virion incorporation of the gH/gL/UL128-131 complex [18]. The two viruses used here, HB15-t178b and BADrUL131-Y4, are both AD169-derived, but while HB15-t178b retains the UL131 mutation and hence fails to express a virion-associated gH/gL/UL128-131 complex, repair of the UL131 gene in BADrUL131-Y4 restores UL131 expression and virion-incorporation of the gH/gL/UL128-131 complex [17].

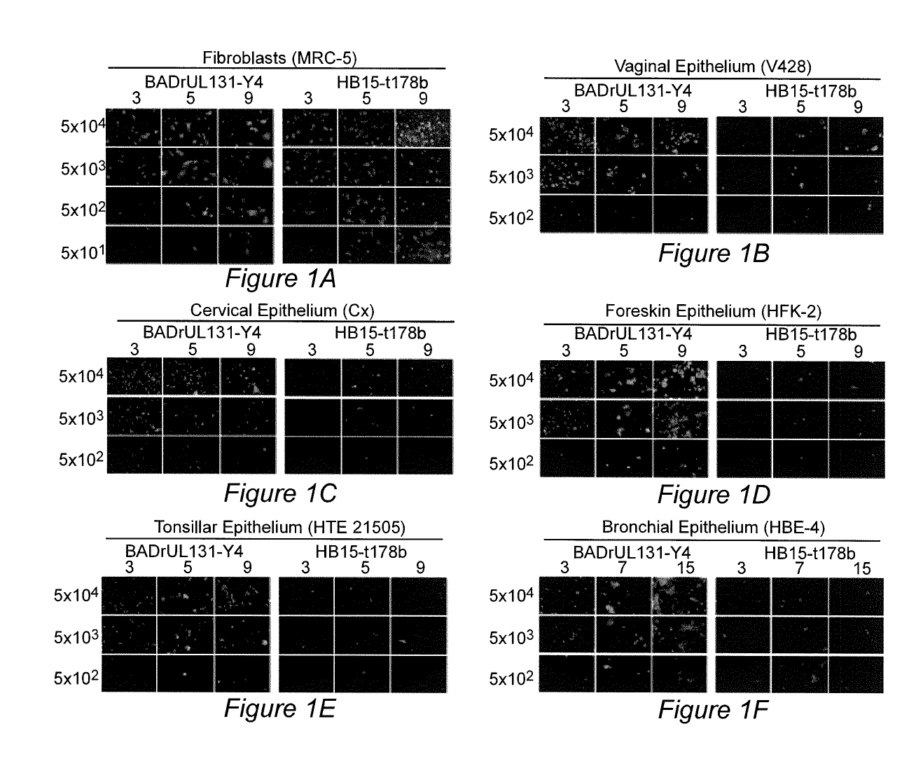

As shown in FIG. 1A, the two viral inocula were well matched for entry into MRC-5 fibroblasts even as the inocula were serially diluted down to low levels. Cells originating from genital mucosal tissues, including vagina, cervix, and foreskin, all displayed a pronounced requirement for gH/gL/UL128-131, as evidenced by high levels of GFP+ cells on day 3 following BADrUL131-Y4 infection and a virtual absence of GFP+ cells from cultures that received matching inocula of HB15-t178b (FIG. 1, panels B-D). Similar data were obtained with airway epithelial cells from tonsil and bronchus (FIG. 1, panels E and F). Foreskin and bronchial epithelial cells appeared to support the full replication cycle of BADrUL131-Y4, resulting in viral spread, as suggested by increased GFP expression in BADrUL131-Y4-infected cell cultures over time (FIG. 1, panels D and F). In contrast, the number of GFP+ cells remained stable over time in BADrUL131-Y4-infected vaginal, cervical, and tonsillar epithelial cells (FIG. 1, panels B, C, and E), suggesting a possible post-entry block to BADrUL131-Y4 replication in these cells.

### 3.2 Peptide Immunogens Elicit Potent Neutralizing Activities in Rabbits

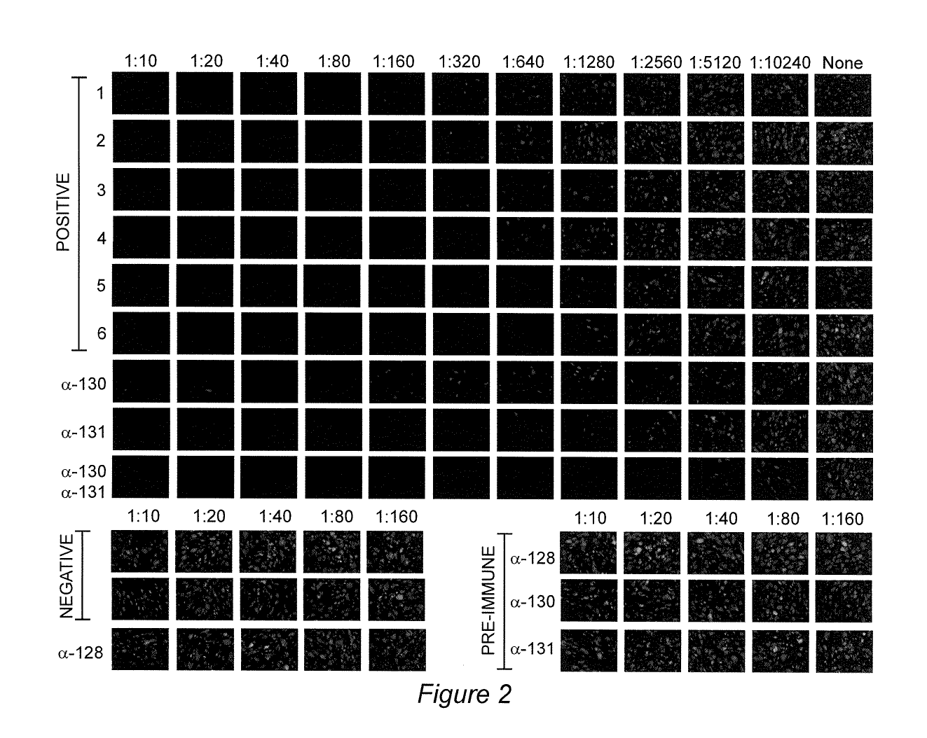

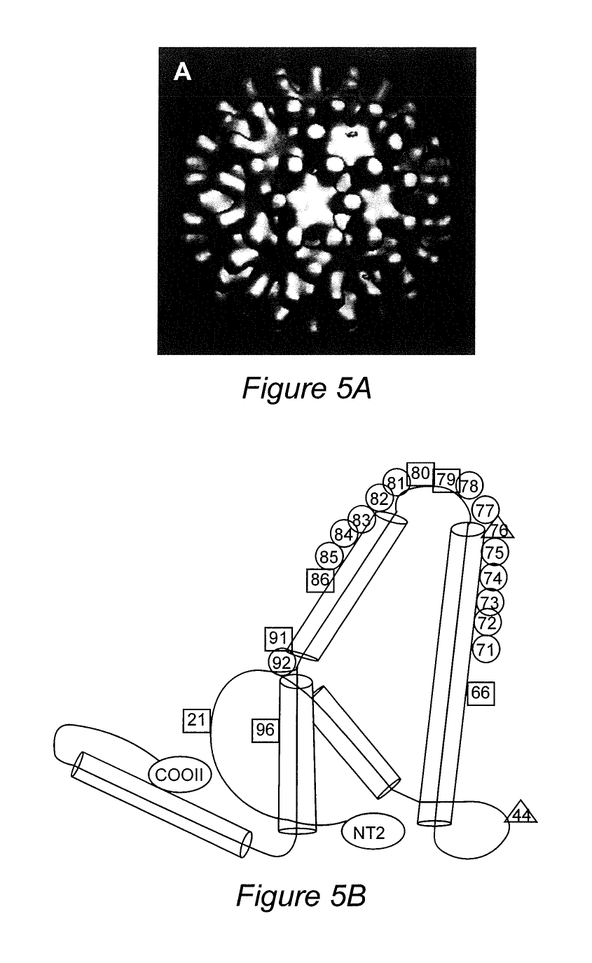

We determined if rabbit sera raised against peptides from UL128, UL130, or UL131 neutralized epithelial cell entry. The rabbit sera were evaluated using a GFP-based neutralizing assay similar to one developed to study sera from naturally infected or experimentally vaccinated humans [12]. Consistent with our previous report [12], sera from two CMV seronegative donors had no effect on epithelial entry, whereas seropositive sera from six naturally infected donors blocked epithelial entry even out to dilutions of 1:640 (FIG. 2). Sera obtained from all three rabbits prior to immunization as well as antiserum to the UL128 peptide failed to neutralize epithelial cell entry at any concentration (FIG. 2). Rabbit antisera to UL130 or UL131 peptides neutralized epithelial entry with activities within the range defined by the seropositive sera; however, a 50:50 mixture of the anti-UL130 and anti-UL131 sera retained neutralizing activity when diluted four-fold higher than the strongest seropositive human serum (FIG. 2). All three rabbit sera failed to neutralize fibroblast entry at any concentration (FIG. 4 and data not shown).

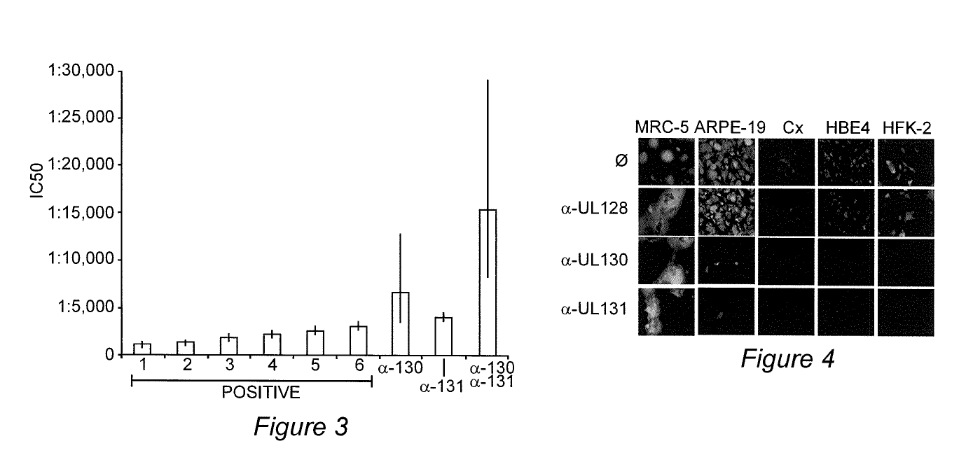

GFP fluorescence was used to calculate neutralizing titers, assessed as IC₅₀ values, for each serum or serum combination (see Materials and Methods). Titers for the six seropositive sera ranged from 1:1007 to 1:3118. Titers for the antiserum to UL130 (1:6732) or UL131 (1:4096) were slightly above the range defined by the seropositive sera, while that of the UL130+UL131 combination (1:15421) was considerably higher (FIG. 3).
3.3 Antibodies to UL130 and UL131 Peptides Neutralize CMV Entry into Epithelial Cells from Mucosal Tissues

To directly confirm that proteins comprising the gH/gL/UL128-131 complex must be physically present on the virion surface to facilitate viral entry into these cells, we determined the ability of rabbit anti-peptide sera to block viral entry. As before, the three antisera had no effect on BADrUL131-Y4 entry into fibroblasts and the anti-UL130 and anti-UL131 sera potently inhibited entry to ARPE-19 epithelial cells while the anti-UL128 serum did not (FIG. 4). That entry into epithelial cells from cervix, foreskin, and bronchus was highly sensitive to neutralization by both the anti-UL130 and the anti-UL131 sera (FIG. 4) physically confirmed that entry into these cell types involves UL130 as well as UL131.

### 3.4 The UL128 and UL131 Peptides are Highly Conserved Among CMV Isolates

Antigenic variation is important for any potential vaccine immunogen. The UL128-131 proteins are known to be highly conserved between CMV strains [23], but to specifically determine amino acid variability within the UL128, UL130, and UL131 peptides, DNA sequences from 29 distinct strains available from GenBank were translated and aligned using ClustalW. Nine amino acid positions in UL128 and three in UL131 were polymorphic, but within the UL128 and UL131 peptide regions the amino acid sequences were 100% identical. UL130 was more variable with 19 polymorphic positions resulting in five variants within the UL130 peptide region, as shown in Table 2. These results suggest that antibodies to the UL131 peptide should cross neutralize the majority of CMV strains, whereas antisera raised against the UL130 peptide might be less effective at neutralizing strains expressing different UL130 variants.

TABLE 2
Polymorphisms within the UL130 peptide

| | | | number of |
|---|---|---|---|
| Variant | SEQ ID NO: | UL30 peptide sequence^{a} | strains |
| 1 | 1 | SWSTLTANQNPSPPWSKLTY^{b} | 2 |
| 2 | 2 | PWSTLTANQNPSPPWSKLTY | 11 |
| 3 | 3 | PWFTLTANQNPSPPWSKLTY | 1 |
| 4 | 4 | PWSTLTANKNPSPPWSKLTY | 6 |
| 5 | 5 | PWSTLTANQNPSPLWSKLTY | 9 |

^{a}Amino acid changes relative to the reference strain are shown in bold and underlined
^{b}Reference strain TR (from which the sequence for the UL130 peptide was derived)

### 4. Discussion

In previous studies we and others observed that sera from CMV-infected humans have two neutralizing activities; one is a moderate activity, comprised mostly of antibodies to gB, that neutralizes viral entry into fibroblasts. The other is more potent and neutralizes viral entry into epithelial cells [7, 12]. The antigen specificities of the latter are unknown, but because both the gH/gL/UL128-131 complex and this neutralizing activity are specific to epithelial cell entry, the antibodies that comprise the epithelial entry neutralizing activity presumably target gH/gL/UL128-131.

Our results further suggest that a vaccine that incorporates gH/gL/UL128-131 epitopes to induce epithelial entry neutralizing activities might be effective at preventing viral acquisition through mucosal epithelia. This presumes, however, that infection of mucosal epithelial cells is gH/gL/UL128-131-mediated and hence neutralizable with gH/gL/UL128-131-specific antibodies. To date, the majority of work on the mechanism and neutralizing activities against epithelial entry have used ARPE-19 cells, which are derived from the retinal pigment epithelium of the eye. The importance of gH/gL/UL128-131 for viral entry has also been confirmed for tumor cells of epithelial origin derived from breast, cervix, lung, and colon [18]. Here, we evaluated CMV entry into cells derived from tissues believed to be most relevant to CMV acquisition—airway and genital mucosa—and in all cases found that entry is gH/gL/UL128-131-dependent. We further observed using a subset of cell lines that entry can be blocked by antibodies to epitopes within the gH/gL/UL128-131 complex. These results support the hypothesis that a vaccine that elicits epithelial entry-specific neutralizing responses in mucosal secretions may provide sterilizing immunity.

Little is known about the neutralizing epitopes within the gH/gL/UL128-131 complex, and of central importance for vaccine design, it remains uncertain whether conformational epitopes unique to the full gH/gL/UL128-131 complex will be required, or whether subunits or even peptides will be sufficient to elicit neutralizing activities comparable to natural infection. Some evidence suggests that neutralizing epitopes may often require multisubunit complexes. A recently described panel of 17 human monoclonals having potent neutralizing activities against epithelial entry predominantly recognize epitopes that require two or more subunits—only one of the 17 antibodies reacted with an individual subunit [8]. In addition, the Towne virus expresses UL128 and UL131, but expression of UL130 is impaired by a C-terminal frame shift that alters the protein's stability and steady-state levels [24]. Yet, despite the presumed ability to express UL128 and UL131 in vivo, the Towne virus does not elicit high titer neutralizing antibodies specific for epithelial entry [12]. This may be because the absence of UL130 results in retention of the remainder of the complex (gH/gL/UL128/UL131) in the endoplasmic reticulum and subsequent failure of this complex to traffic to the cell surface or become incorporated into virions [21]. Thus, for a live attenuated vaccine, UL128 and UL131 are not sufficient. Alternatively, animal antibodies raised against individual UL128, UL130, or UL131 peptides or recombinant proteins do neutralize epithelial or endothelial cell entry, indicating that each subunit contains neutralizing epitopes [5-7]. However, potency of animal antisera relative to human immune sera has not been reported. We observed that peptide epitopes within UL130 or UL131 can elicit epithelial entry neutralizing activities comparable to those induced by natural infection when administered to rabbits using optimal adjuvants. This indicates that the gH/gL/UL128-131 complex contains at least two potent neutralizing epitopes that do not require multisubunit complexes. While the anti-UL128 peptide serum did not neutralize, the peptide used to raise this serum contained an inadvertent isoleucine insertion, and although it retains epitopes sufficient for the antiserum to recognize the native protein [21], the possibility remains that the isoleucine disrupts a neutralizing epitope. Moreover, as UL128, UL130, and UL131 are respectively 171, 235, and 129 amino acids long, significant regions of these proteins have not been evaluated and may contain additional neutralizing epitopes. Indeed, that at least two of the three peptides studied contain neutralizing epitopes suggests that there may be many more.

The rabbit immunization protocol used here was designed to elicit maximal antibody responses and may not be recapitulated in humans. To achieve comparable antibody responses in humans it may be necessary to utilize alternative adjuvants, carriers, or vector systems that are being developed specifically to elicit robust responses to peptide epitopes. Peptide-based ELISAs failed to detect antibodies reactive to these peptides in a small panel of seropositive human sera (Cui and McVoy, unpublished results). However, vaccination may be more effective than infection at eliciting anti-peptide antibody responses, and, given that monoclonals that neutralize this entry pathway are exceedingly potent [8], it is possible that low antibody levels could confer significant neutralizing activities.

The UL130 peptide exhibits strain heterogeneity and thus antibodies to this epitope may not cross-neutralize all CMV strains. However, an instructive implication of our results for vaccine development is that peptide or single subunit immunogens have the potential to produce high titer epithelial entry neutralizing responses, and hence, representation of complex conformational epitopes may not be necessary.

Although theoretically compelling, the premise that epithelial entry neutralizing antibodies can protect against infection is supported mainly by evidence that naturally acquired humoral immunity, which has high epithelial entry neutralizing activity, provides clinical benefits [2, 25, 26], whereas experimental vaccines that induce weak epithelial entry neutralizing responses (compared to natural infection) [12] provide either partial [4] or no protection against primary infection [27]. Thus, our use of seropositive sera as a benchmark for evaluating immunogens is somewhat arbitrary; neutralizing activities comparable to those found in seropositive sera may not provide adequate protection, and while higher levels may be achievable and might enhance protection, other factors, such as cellular immunity or antibodies that neutralize fibroblast entry, may also be important. Ultimately, the importance of epithelial entry neutralizing antibodies for CMV vaccine protection may only be resolved through clinical trials of candidate vaccines that elicit neutralizing activities equivalent or superior to natural infection. The data presented here may aid in development of such candidate vaccines.

### REFERENCES

[1] Snydman D R. Historical overview of the use of cytomegalovirus hyperimmune globulin in organ transplantation. Transpl Infect Dis 2001; 3 Suppl 2:6-13.
[2] Nigro G, Adler S P, La Tone R, Best A M. Passive Immunization during Pregnancy for Congenital Cytomegalovirus Infection. N Engl J Med 2005; 353(13):1350-62.
[3] Plotkin S A, Starr S E, Friedman H M, Gonczol E, Weibel R E. Protective effects of Towne cytomegalovirus vaccine against low-passage cytomegalovirus administered as a challenge. J Infect Dis 1989; 159(5):860-5.
[4] Pass R F, Zhang C, Evans A, Simpson T, Andrews W, Huang M L, et al. Vaccine prevention of maternal cytomegalovirus infection. N Engl J Med 2009 Mar. 19; 360(12):1191-9.
[5] Wang D, Shenk T. Human cytomegalovirus virion protein complex required for epithelial and endothelial cell tropism. Proc Natl Acad Sci USA 2005 Dec. 13; 102(50):18153-8.
[6] Adler B, Scrivano L, Ruzcics Z, Rupp B, Sinzger C, Koszinowski U. Role of human cytomegalovirus UL131A in cell type-specific virus entry and release. J Gen Virol 2006 September; 87(Pt 9):2451-60.
[7] Gema G, Sarasini A, Patron M, Percivalle E, Fiorina L, Campanini G, et al. Human cytomegalovirus serum neutralizing antibodies block virus infection of endothelial/epithelial cells, but not fibroblasts, early during primary infection. J Gen Virol 2008 April; 89(Pt 4):853-65.
[8] Macagno A, Berasconi N L, Vanzetta F, Dander E, Sarasini A, Revello M G, et al. Isolation of human monoclonal antibodies that potently neutralize human cytomegalovirus infection by targeting different epitopes on the gH/gL/UL128-131A complex. J Virol 2010 January; 84(2):1005-13.
[9] Hahn G, Revello M G, Patrone M, Percivalle E, Campanini G, Sarasini A, et al. Human cytomegalovirus UL131-128 genes are indispensable for virus growth in endothelial cells and virus transfer to leukocytes. J Virol 2004 September; 78(18):10023-33.
[10] Ryckman B J, Jarvis M A, Drummond D D, Nelson J A, Johnson D C. Human cytomegalovirus entry into epithelial and endothelial cells depends on genes UL128 to UL150 and occurs by endocytosis and low-pH fusion. J Virol 2006 January; 80(2):710-22.
[11] Dargan D J, Douglas E, Cunningham C, Jamieson F, Stanton R J, Baluchova K, et al. Sequential mutations associated with adaptation of human cytomegalovirus to growth in cell culture. J Gen Virol 2010 June; 91(Pt 6):1535-46.
[12] Cui X, Meza B P, Adler S P, McVoy M A. Cytomegalovirus vaccines fail to induce epithelial entry neutralizing antibodies comparable to natural infection. Vaccine 2008 Aug. 19; 26:5760-6.
[13] Sauer A, Wang J B, Hahn G, McVoy M A. A human cytomegalovirus deleted of internal repeats replicates with near wild type efficiency but fails to undergo genome isomerization. Virology 2010 May 25; 401(1):90-5.
[14] Hobom U, Brune W, Messerle M, Hahn G, Koszinowski UH. Fast screening procedures for random transposon libraries of cloned herpesvirus genomes: mutational analysis of human cytomegalovirus envelope glycoprotein genes. J Virol 2000; 74(17):7720-9.
[15] Hahn G, Jarosch M, Wang J B, Berbes C, McVoy M A. Tn7-mediated introduction of DNA sequences into bacmid-cloned cytomegalovirus genomes for rapid recombinant virus construction. J Virol Methods 2003 February; 107(2):185-94.
[16] Yu D, Smith G A, Enquist L W, Shenk T. Construction of a self-excisable bacterial artificial chromosome containing the human cytomegalovirus genome and mutagenesis of the diploid TRL/IRL13 gene. J Virol 2002; 76(5):2316-28.
[17] Wang D, Bresnahan W, Shenk T. Human cytomegalovirus encodes a highly specific RANTES decoy receptor. Proc Natl Acad Sci USA 2004 Nov. 23; 101(47):16642-7.
[18] Wang D, Shenk T. Human cytomegalovirus UL131 open reading frame is required for epithelial cell tropism. J Virol 2005 August; 79(16):10330-8.
[19] Cui X, McGregor A, Schleiss M R, McVoy M A. Cloning the complete guinea pig cytomegalovirus genome as an infectious bacterial artificial chromosome with excisable origin of replication. J Virol Methods 2008 May; 149(2):231-9.
[20] Zabner J, Karp P, Seiler M, Phillips S L, Mitchell C J, Saavedra M, et al. Development of cystic fibrosis and noncystic fibrosis airway cell lines. Am J Physiol Lung Cell Mol Physiol 2003 May; 284(5):L844-54.
[21] Ryckman B J, Rainish B L, Chase M C, Borton J A, Nelson J A, Jarvis M A, et al. Characterization of the human cytomegalovirus gH/gL/UL128-131 complex that mediates entry into epithelial and endothelial cells. J Virol 2008 January; 82(1):60-70.
[22] Ryckman B J, Chase M C, Johnson D C. Human cytomegalovirus TR strain glycoprotein O acts as a chaperone promoting gH/gL incorporation into virions but is not present in virions. J Virol 2010 March; 84(5):2597-609.
[23] Baldanti F, Paolucci S, Campanini G, Sarasini A, Percivalle E, Revello M G, et al. Human cytomegalovirus UL131A, UL130 and UL128 genes are highly conserved among field isolates. Arch Virol 2006 June; 151(6):1225-33.
[24] Patrone M, Secchi M, Fiorina L, Ierardi M, Milanesi G, Gallina A. Human cytomegalovirus UL130 protein promotes endothelial cell infection through a producer cell modification of the virion. J Virol 2005 July; 79(13):8361-73.
[25] Adler S P, Chandrika T, Lawrence L, Baggett J. Cytomegalovirus infections in neonates acquired by blood transfusions. Pediatr Infect Dis 1983 March-April; 2(2):114-8.
[26] Yeager A S, Grumet F C, Hafleigh E B, Arvin A M, Bradley J S, Prober C G. Prevention of transfusion-acquired cytomegalovirus infections in newborn infants. J Pediatr 1981 February; 98(2):281-7.
[27] Adler S P, Starr S E, Plotkin S A, Hempfling S H, Buis J, Manning M L, et al. Immunity induced by primary human cytomegalovirus infection protects against secondary infection among women of childbearing age [published erratum appears in J Infect Dis 1995 April; 171(4):1080]. J Infect Dis 1995; 171(1):26-32.

### Example 2

### An HBC-Vectored Peptide-Based Cytomegalovirus Vaccine

Human cytomegalovirus (CMV) is the major infectious cause of birth defects in the United States and worldwide. Recent demonstration that the glycoprotein B (gB)/MF59 vaccine has 50% efficacy in protecting women against primary CMV infection is a landmark in CMV vaccine research. However, 50% efficacy may be insufficient for vaccine licensure. Thus, one challenge is to determine what can be added to a gB-based vaccine to increase efficacy to an acceptable level. Recent work has shown that CMV seropositive people have high levels of antibodies that neutralize viral entry into epithelial cells and that comparable levels are not achieved by the gB/MF59 vaccine. Epithelial entry-specific neutralizing epitopes reside within a virion glycoprotein complex consisting of gH, gL, UL128, UL130, and UL131 (gH/gL/UL128-131). Example 1 describes the identification of two peptide sequences, one from UL130 and one from UL131, that are capable of eliciting potent epithelial entry-specific neutralizing responses. This Example describes a platform for eliciting antibody responses to peptide epitopes by engineering the desired peptides into an external loop of the hepatitis virus B core antigen (HBcAg) protein. The modified proteins self-assemble into virus-like particles (VLPs), which serve as potent immunogens and elicit strong antibody responses to the inserted peptide epitopes. Accordingly, chimeric HBcAg proteins that contain the UL130 and UL131 peptide epitopes are engineered in this manner and evaluated for their ability to elicit epithelial entry-specific neutralizing activities in mice. Optimal chimeric VLPs are further evaluated for compatibility with the gB subunit vaccine. The results of the proposed studies provide a novel vaccine strategy for advancement to clinical development.

This example thus describes strategies for constructing, expressing, and purifying chimeric HBcAg proteins containing two CMV peptides that are known to induce potent epithelial entry neutralizing antibodies, and the use of these proteins to immunize mice and evaluate their ability to (1) induce antibodies that neutralize CMV infection of epithelial cells to levels comparable to those of sera from naturally infected human subjects, and (2) work in conjunction with recombinant gB to elicit both fibroblast and epithelial entry neutralizing responses.

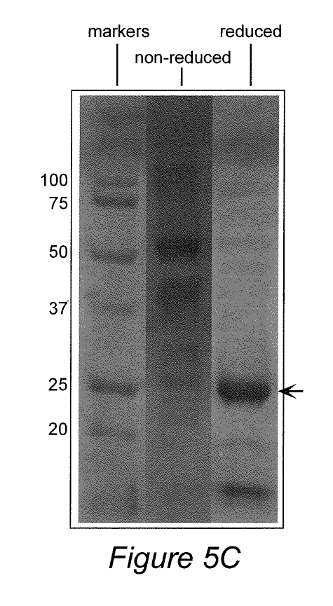

The strategy that is utilized takes advantage of the intrinsic immunogenicity of hepatitis B virus (HBV) core protein (HBcAg). This protein, which is the structural component of the virus nucleocapsid, assembles into particles that are highly immunogenic during natural HBV infection. The protein is relatively small (Mw=21000) and spontaneously assembles into icosahedral structures (FIG. 5A) containing 120 HBcAg dimers. X-ray crystallographic studies have shown that the dimers form “spikes” on the surface of the particles composed of 4 alpha helices connected by a loop. FIG. 5A shows the particle structure deduced from these studies, while FIG. 5B is a depiction of the monomer structure within this particle. We and others have shown that the major anti-HBV core antibody binding sites are localized to these spikes.

The particle structure confers high immunogenicity to foreign epitopes which can be incorporated into the particles by chemical coupling or by inclusion of the appropriate nucleotides into the coding sequence for the protein. Although HBV was the first of this class of the Hepadnaviruses identified, several additional ones are now known. These include the woodchuck hepatitis B virus, the ground squirrel hepatitis B virus, and the duck hepatitis B virus. Each of the viruses has its unique core protein. Although these proteins are antigenically distinct, each exhibits the same physical properties of self-assembly, overall particle structure, high immunogenicity, and the ability to confer high immunogenicity to foreign epitopes. Moreover, all of these proteins are easily expressed and purified from bacterial expression systems.

Because many people may have antibodies to HBV, there is concern that pre-existing antibodies to the human HBcAg could compromise the effectiveness of vaccines based on this protein. Therefore, it would be advantageous to use the rodent or bird core proteins as the carrier. For this reason, we have studied the structures of these core proteins and identified the optimum positions for insertion of foreign epitopes, thereby creating useful vaccine platforms (see, for example, U.S. Pat. Nos. 6,887,464 (Coleman and Peterson); 7,279,555 (Peterson) and 6,231,864 (Birkett), the complete contents of each of which are hereby incorporated by reference). Using these platforms we have demonstrated the production of high titer anti-epitope antibodies in immunized animals using a variety of different epitopes. Therefore, the two peptides identified above are excellent candidates for evaluation as vaccine components in the context of the HBcAg platform. E. coli expression vectors encoding woodchuck HBcAg (WHBcAg) or duck HBcAg (DHBcAg) with the UL130 or UL131 peptide sequences engineered into the appropriate immunogenic location have been constructed. For example, chimeric protein BEE6 was genetically engineered by modifying the nucleic acid sequence encoding WHBcAg to incorporate the UL130 peptide sequence inserted after residue position 84 (FIG. 6). BEE6 was expressed in E. coli using standard techniques and purified in vitro. SDS-PAGE analysis indicates that under reducing conditions the protein migrates as a relatively pure monomer with an apparent molecular weight consistent with the 25 kDa molecular weight theoretically predicted from the amino acid sequence (FIG. 5C). That BEE6 forms higher molecular weight species under nonreducing conditions (FIG. 5C) indicates that disulfide bonds form between monomers, consistent with particle assembly. Light scattering indicates that the average particle diameter is 39 nm, consistent with particles formed from wild type WHBcAg. Thus, BEE6 appears to assemble efficiently into VLPs.

Other chimeric proteins, such as the UL130 peptide in the DHBcAg platform or the UL131 peptide in either DHBcAg or WHBcAg platforms are under development. Substitutions of the amino acids on either side of the peptide insertions are often necessary to achieve proper folding and particle assembly. All proteins are expressed in E. coli and purified in mg quantities sufficient for immunization studies.

Animal immunizations. Groups of 10 Balb/c mice are immunized with BEE6 or other chimeric proteins using either Freund's or alum adjuvants subQ into the scuff of the neck. Animals are boosted at week 3 and again at week 6. At week 9 animals are sacrificed and terminal blood draws obtained. Initial studies of each protein individually inform subsequent studies. Based on serological evaluations (below), the optimal HBcAg (woodchuck vs Duck) is selected for each peptide.

The second immunization study evaluates three combinations: (1) the two optimal HBcAg-peptide proteins administered together; (2) two optimal HBcAg-peptide proteins administered with recombinant gB; (3) recombinant gB alone. These studies establish whether there are any competitive/inhibitory effects that arise from combining two or three immunogens as compared to each immunogen administered separately.

Serological evaluations. The key readout is CMV neutralizing antibody titers using both epithelial and fibroblast cells. Mouse sera are compared directly to known high titer human sera for ability to neutralize CMV entry. Antibodies reactive to the respective peptides are measured via western blot and ELISA assays. Western blot antigens include native UL130, UL131, and gB proteins in CMV-infected cell lysates and the purified chimeric HBcAg and gB proteins. For these studies the heterologous HBcAg proteins serve as negative controls—i.e., HBcAg-UL130 serves as a negative control for detection of antibodies to HBcAg-UL131, etc. For ELISA assays 96-well plates are coated with gB or synthetic UL130 and UL131 peptides (identical to the sequences inserted into the HBcAgs) and incubated with dilutions of mouse sera. Immobilized mouse IgG are detected with HRP-conjugated anti-mouse IgG followed by colorimetric HRP substrate reaction.

Alternative western blot antigens include lysates of 293T cells transfected with expression vectors, 293T cells infected with adenovirus vectors, and insect cells infected with baculovirus vectors. All three proteins can be readily detected in these samples by western using rabbit antisera. Moreover, westerns using transfected 293T cell lysates were recently used to detect anti-UL130 antibodies in human sera (Saccoccio, F. M., M. K. Gallagher, S. P. Adler, and M. A. McVoy. 2011. Neutralizing Activity of Saliva against Cytomegalovirus. Clinical and Vaccine Immunology, 18:1536-1542).

gB adjuvanted with MF59 produces high titer fibroblast entry neutralizing responses. HBcAg-peptide immunogens, when added to recombinant gB, result in a vaccine regimen that elicits antibody responses that match or exceed the levels of fibroblast and epithelial cell entry neutralizing antibodies that are found in convalescent human sera. The demonstration that a novel gB/HBcAg-peptide vaccine matches or exceeds both types of neutralizing activity provides compelling data for the pursuit of further refinements, such as additional epitope discovery and the use of duck HBcAg for the first two doses and woodchuck HBcAg for the third dose (to minimize antibody-mediated clearance of the immunogen); as well as optimization of dosage, regimen (timing and number of doses), and formulation (ratios of each component); and also confirmation of immunogenicity in larger outbred animals (e.g., rabbits, guinea pigs). Protection studies of a human CMV vaccine are performed in the context of clinical trials.

### Example 3

### Immunization of Mice with CMV Peptides

Murine antisera are produced against peptides derived from proteins UL130 and UL131. Immunization is conducted by a red blood cell-mediated delivery to the liver and spleen, where they are processed by antigen-presenting cells. A RBC-targeting fusion protein (FP) is used for this purpose. The FP is comprised of a single chain variable fragment (scFv) of a monoclonal antibody (Mab), TER-119, which binds murine glycophorin A, fused to core streptavidin (Adekar S P, Segan AT, Chen C, Bermudez R, Elias M D, Selling B H, Kapadnis B P, Simpson L L, Simon P M, Dessain S K. 2011. Enhanced neutralization potency of botulinum neurotoxin antibodies using a red blood cell-targeting fusion protein. PLoS One. 6(3):e17491. PMID: 21399689). The FP is tetrameric so the immunizing material is composed of a peptide:FP molar ratio of 4:1 in order to occupy the 4 biotin-binding sites. Peptides are synthesized with a C-terminal biotin-Lys residue (GenScript, Piscataway, N.J.). Groups of mice (Balb/c) are immunized with 1.5 μg of biotinylated peptide: FP complexes i.v., s.c. or i.m. and boosted 2 and 4 weeks without additional adjuvants. Control groups include mice immunized with peptides conjugated to KLH and emulsified with the adjuvant TiterMax (administered s.c.), mice given peptide alone, mice given FP alone and mice receiving buffer. Sera are collected on week 5 and are analyzed by ELISA using peptide-coated plates and goat anti-mouse-HRP. High titer anti-peptide murine antisera are tested for their ability to inhibit entry of a GFP-tagged CMV into human epithelial cells. Inhibition of entry is detected by a reduction in the number of GFP+ cells in the culture (compared to cells infected with untreated virus) and reduction in net GFP expression for the culture, as described in Example 1.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

We claim:

1. A combination peptide, polypeptide or protein comprising
I. a plurality of copies of one or both of
a. amino acid residues 27-46 of a UL130 cytomegalovirus (CMV) protein; and
b. amino acid residues 90-106 of a UL131 CMV protein;
or
II. i) one or more copies of one or both of
a. amino acid residues 27-46 of a UL130 CMV protein; and
b. amino acid residues 90-106 of a UL131 CMV protein; and
ii) an additional proteinaceous entity,
wherein said combination peptide, polypeptide or protein is not full-length UL130 CMV or full-length UL131 protein.

2. The combination peptide, polypeptide or protein of claim 1, wherein said combination peptide, polypeptide or protein comprises a sequence:
(SEQ ID NO: 7)
X₁WX₂TLTANX₃NPSPPWSKLTY
wherein
X₁=S or P;
X₂=S or F; and
X₃=Q or K.

3. The combination peptide, polypeptide or protein of claim 2, wherein said combination peptide, polypeptide or protein comprises an amino acid sequence selected from the group consisting of: SWSTLTANQNPSPPWSKLTY (SEQ ID NO: 1); PWSTLTANQNPSPPWSKLTY (SEQ ID NO: 2); PWFTLTANQNPSPPWSKLTY (SEQ ID NO:3); PWSTLTANKNPSPPWSKLTY (SEQ ID NO:4); and PWSTLTANQNPSPLWSKLTY (SEQ ID NO: 5).

4. The combination peptide, polypeptide or protein of claim 1, wherein said combination peptide, polypeptide or protein comprises an amino acid sequence SDFRRQNRRGGTNKRTT (SEQ ID NO: 6).

5. The combination peptide, polypeptide or protein of claim 1, wherein said additional proteinaceous entity is selected from the group consisting of: a carrier protein suitable for administration to humans, a recombinant hepatitis B core protein, and a red blood cell targeting protein.

6. The combination peptide, polypeptide or protein of claim 1, further comprising linker or spacer sequence located between said copies of said one or both of amino acid residues 27-46 of a UL130 CMV protein and amino acid residues 90-106 of a UL131 CMV protein.

7. A composition for eliciting a neutralizing immune response against cytomegalovirus in a human subject in need thereof, said composition comprising at least one combination peptide, polypeptide or protein comprising:
I. a plurality of copies of one or both of
a. amino acid residues 27-46 of a UL130 cytomegalovirus (CMV) protein; and
b. amino acid residues 90-106 of a UL131 CMV protein;
or
II. i) one or more copies of one or both of
a. amino acid residues 27-46 of a UL130 CMV protein; and
b. amino acid residues 90-106 of a UL131 CMV protein; and
ii) an additional proteinaceous entity;
and
a physiologically compatible carrier.

8. The composition of claim 7, wherein said combination peptide, polypeptide or protein comprises a sequence:
(SEQ ID NO: 7)
X₁WX₂TLTANX₃NPSPPWSKLTY
wherein
X₁=S or P;
X₂=S or F; and
X₃=Q or K.

9. The composition of claim 7, wherein said combination peptide, polypeptide or protein comprises an amino acid sequence selected from the group consisting of:
(SEQ ID NO: 1)
SWSTLTANQNPSPPWSKLTY;
(SEQ ID NO: 2)
PWSTLTANQNPSPPWSKLTY;
(SEQ ID NO: 3)
PWSTLTANQNPSPPWSKLTY;
(SEQ ID NO: 4)
PWSTLTANKNPSPPWSKLTY;
and
(SEQ ID NO: 5)
PWSTLTANQNPSPLWSKLTY.

10. The composition of claim 7, wherein said combination peptide, polypeptide or protein comprises an amino acid sequence SDFRRQNRRGGTNKRTT (SEQ ID NO: 6).

11. The composition of claim 7, wherein said additional proteinaceous entity is selected from the group consisting of: a carrier protein suitable for administration to humans, a recombinant hepatitis B core protein, and a red blood cell targeting protein.

12. The composition of claim 7, further comprising linker or spacer sequence located between said copies of said one or both of amino acid residues 27-46 of a UL130 CMV protein and amino acid residues 90-106 of a UL131 CMV protein.

13. The composition of claim 7, further comprising CMV glycoprotein B or a genetically engineered version thereof.

14. The composition of claim 7, further comprising an adjuvant.

15. A method of eliciting a neutralizing immune response against cytomegalovirus (CMV) in a human subject in need thereof, comprising the step of
administering to said human subject a composition comprising at least one combination peptide, polypeptide or protein comprising
I. a plurality of copies of one or both of
a. amino acid residues 27-46 of a UL130 CMV protein; and
b. amino acid residues 90-106 of a UL131 CMV protein;
or
II. i) one or more copies of one or both of
a. amino acid residues 27-46 of a UL130 CMV protein; and
b. amino acid residues 90-106 of a UL131 CMV protein; and
ii) an additional proteinaceous entity;
and
a physiologically compatible carrier.

16. The method of claim 15, wherein said combination peptide, polypeptide or protein comprises a sequence:
(SEQ ID NO: 7)
X₁WX₂TLTANX₃NPSPPWSKLTY
wherein
X₁=S or P;
X₂=S or F; and
X₃=Q or K.

17. The method of claim 15, wherein said combination peptide, polypeptide or protein comprises an amino acid sequence selected from the group consisting of:
(SEQ ID NO: 1)
SWSTLTANQNPSPPWSKLTY;
(SEQ ID NO: 2)
PWSTLTANQNPSPPWSKLTY;
(SEQ ID NO: 3)
PWFTLTANQNPSPPWSKLTY;
(SEQ ID NO: 4)
PWSTLTANKNPSPPWSKLTY;
and
(SEQ ID NO: 5)
PWSTLTANQNPSPLWSKLTY.

18. The method of claim 15, wherein said combination peptide, polypeptide or protein comprises an amino acid sequence SDFRRQNRRGGTNKRTT (SEQ ID NO: 6).

19. The method of claim 15, wherein said additional proteinaceous entity is selected from the group consisting of: a carrier protein suitable for administration to humans, a recombinant hepatitis B core protein, and a red blood cell targeting protein.

20. The method of claim 15, further comprising linker or spacer sequence located between said copies of said one or both of amino acid residues 27-46 of a UL130 CMV protein and amino acid residues 90-106 of a UL131 CMV protein.

21. The method of claim 15, wherein said composition further comprises CMV glycoprotein B or a genetically engineered version thereof.

22. The composition of claim 15, wherein said composition further comprises an adjuvant.

23. The method if claim 15, wherein said immune response is production of neutralizing antibodies against CMV.

24. The method of claim 23, wherein neutralizing antibodies prevent entry of CMV into epithelial cells.

25. The method of claim 24, wherein said epithelial cells are oral or genital mucosal epithelial cells.

26. A method of preventing cytomegalovirus (CMV) entry into cells, comprising the step of
exposing said CMV to neutralizing antibodies which bind specifically to one or both of
i) one or more epitopes within amino acid residues 27-46 of a UL130 CMV protein; and
ii) one or more epitopes within amino acid residues 90-106 of a UL131 CMV protein.

27. A nucleic acid vaccine composition for vaccinating a subject against cytomegalovirus (CMV), comprising
i) a nucleic acid expression system comprising a nucleic acid that encodes at least one copy of one or more of:
a peptide, polypeptide or protein comprising amino acid residues 27-46 of a UL130 CMV protein, or a functional variant thereof; and
a peptide, polypeptide or protein comprising amino acid residues 90-106 of a UL131 CMV protein, or a functional variant thereof;
said nucleic acid being operably linked to a promoter;
and
ii) a physiologically acceptable carrier.

28. The nucleic acid vaccine composition of claim 27, wherein said nucleic acid is selected from DNA and RNA.

29. The nucleic acid vaccine composition of claim 27, wherein said nucleic acid expression system is a recombinant plasmid vector.

30. The nucleic acid vaccine composition of claim 27, wherein said nucleic acid expression system is a recombinant viral or bacterial expression vector.

31. A method of generating neutralizing antibodies against cytomegalovirus (CMV), comprising the step of
administering to an antibody producing mammal, a composition comprising one or more polypeptides comprising
I. a plurality of copies of one or both of
a. amino acid residues 27-46 of a UL130 CMV protein; and
b. amino acid residues 90-106 of a UL131 CMV protein;
or
II. i) one or more copies of one or both of
a. amino acid residues 27-46 of a UL130 CMV protein; and
b. amino acid residues 90-106 of a UL131 CMV protein; and
ii) an additional proteinaceous entity;
and
a physiologically compatible carrier.

32. A cytomegalovirus (CMV) neutralizing antibody generated by administering, to an antibody producing mammal, a composition comprising one or more polypeptides comprising
I. a plurality of copies of one or both of
a. amino acid residues 27-46 of a UL130 CMV protein; and
b. amino acid residues 90-106 of a UL131 CMV protein;
or
II. i) one or more copies of one or both of
a. amino acid residues 27-46 of a UL130 CMV protein; and
b. amino acid residues 90-106 of a UL131 CMV protein; and
ii) an additional proteinaceous entity;
and
a physiologically compatible carrier.


SEQUENCE LISTING (9 sequences)

SEQ ID NO: 1 - protein, 20 aa, Cytomegalovirus
SWSTLTANQNPSPPWSKLTY

SEQ ID NO: 2 - protein, 20 aa, Cytomegalovirus
PWSTLTANQNPSPPWSKLTY

SEQ ID NO: 3 - protein, 20 aa, Cytomegalovirus
PWFTLTANQNPSPPWSKLTY

SEQ ID NO: 4 - protein, 20 aa, Cytomegalovirus
PWSTLTANKNPSPPWSKLTY

SEQ ID NO: 5 - protein, 20 aa, Cytomegalovirus
PWSTLTANQNPSPLWSKLTY

SEQ ID NO: 6 - protein, 17 aa, Cytomegalovirus
SDFRRQNRRGGTNKRTT

SEQ ID NO: 7 - protein, 20 aa, Artificial Sequence
  Synthetic consensus sequence
XWXTLTANXNPSPPWSKLTY

SEQ ID NO: 8 - protein, 216 aa, Artificial Sequence
  Synthetic fusion protein
MDIDPYKEFGSSYQLLNFLPLDFFPDLNALVDTATALYEEELTGREHCSPHHTAIRQALVCWDELTKLIAWMSSNITSGIPEDESWSTLTANQNPSPPWSKLTYEEGTVRTIIVNHVNDTWGLKVRQSLWFHLSCLTFGQHTVQEFLVSFGVWIRTPAPYRPPNAPILSTLPEHTVIRRRGGARASRSPRRRTPSPRRRRSQSPRRRRSQSPSANC

SEQ ID NO: 9 - DNA, 651 bp, Artificial Sequence
  Synthetic nucleic acid sequence encoding fusion protein
atggacatagatccgtataaagagtttggctcctcctatcagctgctgaacttcctgccgctggattttttcccggatctgaatgcgctggttgataccgccaccgcgctgtatgaagaagaactgacgggtcgtgaacactgctcccctcaccatactgctattcgtcaggccctggtgtgctgggacgaactgactaaactgatcgcctggatgagctccaacattacgtctggaattccggaggacgagtcctggtccaccctgaccgctaaccagaacccgtccccgccgtggtccaaactgacctacgaggagggtaccgtaagaacaatcattgtaaatcatgtcaatgatacctggggacttaaggtgagacaaagtttatggtttcatttgtcatgtctcactttcggacaacatacagttcaagaatttttagtaagttttggagtatggatcaggactccagctccatatagacctcctaatgcacccattctctcgactcttccggaacatactgttattcgccgtcgcggtggtgcacgtgcttctcgcagcccacgtcgtcgcactccatctccgcgccgtcgtagatctcaatctccgcgtcgccgtcgctctcaatctccatctgccaactgctga